(19) United States
(12) Patent Application Publication
(10) Pub. No.: US 2013/0295111 A1
(43) Pub. Date: Nov. 7, 2013

(54) METHODS AND COMPOSITIONS FOR TREATING DISORDERS ASSOCIATED WITH HYPERACTIVE IMMUNE SYSTEM

(71) Applicants: Rosa Ana Gonzalez-Quintial, San Diego, CA (US); Roberto G. Baccala, San Diego, CA (US); Brian R. Lawson, San Diego, CA (US); Argyrios N. Theofilopoulos, San Diego, CA (US)

(72) Inventors: Rosa Ana Gonzalez-Quintial, San Diego, CA (US); Roberto G. Baccala, San Diego, CA (US); Brian R. Lawson, San Diego, CA (US); Argyrios N. Theofilopoulos, San Diego, CA (US)

(73) Assignee: The Scripps Research Institute, La Jolla, CA (US)

(21) Appl. No.: 13/816,121

(22) PCT Filed: Aug. 9, 2011
(86) PCT No.: PCT/US11/01405

§ 371 Date: Apr. 5, 2013

Related U.S. Application Data
(60) Provisional application No. 61/401,235, filed on Aug. 10, 2010.

(51) Int. Cl. A61K 39/395; A61K 31/713

(52) U.S. Cl. CPC A61K 39/3955; A61K 31/713
USPC 424/158.1; 514/44 A

(57) ABSTRACT

The present invention provides methods and compositions for treating disorders associated with hyperactive immune responses. The invention also provides methods for lithibiting activation of CD4⁺ T cells and/or suppressing proliferation other overactivated T cells (e.g., CD4CD8 T cells). These methods typically involve the administration to the subject a therapeutically effective amount of a compound that lithibits or suppresses IL-7 signaling or IL- 15 signaling.

### CROSS-REFERENCE TO RELATED APPLICATIONS

The subject patent application claims the benefit of priority to U.S. Provisional Patent Application No. 61/401,235 (filed Aug. 10, 2010). The full disclosure of the priority application is incorporated herein by reference in its entirety and for all purposes.

### STATEMENT OF GOVERNMENT SUPPORT

This invention was made with government support under Contract number R01 AR31203 awarded by the National Institutes of Health. The government has certain rights in the invention.

### BACKGROUND OF THE INVENTION

Excessive inflammation caused by abnormal recognition of host tissue as foreign or prolongation of the inflammatory process may lead to autoimmune or inflammatory diseases as diverse as asthma, diabetes, arteriosclerosis, cataracts, reperfusion injury, and cancer, to post-infectious syndromes such as in infectious meningitis, rheumatic fever, and to rheumatic diseases such as systemic lupus erythematosus and rheumatoid arthritis. The centrality of the inflammatory response in these varied disease processes makes its regulation a major element in the preventive control or cure of many human diseases. Although abnormal inflammatory response may be modulated by anti-inflammatory agents such as corticosteroids, immunosuppressants, non steroidal anti-inflammatory drugs (NSAID), COX-2 inhibitors and protease inhibitors, many of these agents have significant side effects. Corticosteroids may induce Cushingoid features, skin thinning, increased susceptibility to infection and suppression of the hypothalamic-pituitary-adrenal axis. Immunosuppressants may induce hypertension and nephrotoxicity.

T cells are key constituents of the adaptive immune system. Through their T cell receptors (TCR) they recognize peptide-antigens bound to major histocompatibility complex (MHC) proteins on the surface of antigen-presenting cells (APCs). Following TCR-induced activation and expansion, T cells differentiate into several subsets with specific effector roles. The T cell repertoire, however, also contains cells that may react strongly to MHC-bound self-peptides. To prevent autoimmunity, central and peripheral tolerance mechanisms restrict T cell activity, but in certain circumstances a breakdown in these checkpoints may lead to a wide-spectrum of systemic and organ-specific autoimmune diseases. Therefore, the development of therapies that specifically eliminate self-reactive T cells, while preserving those responding to foreign antigens, is a major area of autoimmune research. The involvement of diverse and undefined autoantigens, epitope spreading, and attraction of bystander cells to inflammatory sites, however, have hindered the development of such therapies. As a result, available treatments are broadly immunosuppressive and often associated with significant adverse effects.

There is a need in the art for better means for treating and preventing autoimmune and inflammatory diseases and conditions. The present invention addresses this and other needs.

### SUMMARY OF THE INVENTION

In one aspect, the invention provides methods for treating or alleviating the symptoms of a disorder associated with hyperactive immune response in a subject. These methods involve administering to the subject a therapeutically effective amount of a compound that inhibits or suppresses IL-7 signaling or IL-15 signaling. In some of the methods, the administered compound inhibits or suppresses IL-7 signaling. For example, the compound (e.g., an inhibitory polynucleotide) can down-regulate expression or cellular level of IL-7. The compound can also be one that blocks signaling activity of IL-7 receptor, e.g., an antagonist antibody specific for IL-7Rα. Some of the methods are specifically directed to treating subjects with autoimmune diseases (e.g., lupus or multiple sclerosis).

In another aspect, the invention provides methods for inhibiting activation of CD4⁺ T cells in a subject. These methods entail administering to the subject a therapeutically effective amount of a compound that inhibits or suppresses IL-7 signaling. The compound employed in these methods include compounds that down-regulate expression or cellular level of IL-7 (e.g., inhibitory polynucleotide's) and compounds that block signaling activities of IL-7 receptor (e.g., antagonist antibodies specific for IL-7Rα). These methods are suitable for treating subjects suffering from inflammatory disorders such as multiple sclerosis. The methods can also be employed for preventing relapse of multiple sclerosis in the subject.

In a related aspect, the invention provides methods for suppressing or inhibiting proliferation of an overactivated T cell (e.g., CD4⁻CD8⁻ T cell) in a subject. The methods entail administering to the subject a therapeutically effective amount of a compound that inhibits or suppresses IL-7 signaling or IL-15 signaling. Some of the methods employ a compound that inhibits or suppresses IL-7 signaling. The administered compound can down-regulate expression or cellular level of IL-7. For example, the compound can be an inhibitory polynucleotide such as a siRNA specific for IL-7. Some other methods utilize a compound that blocks signaling activities of IL-7 receptor. For example, an antagonist antibody specific for IL-7Rα can be used in these methods. Some of the methods are directed to treating subjects suffering from autoimmune diseases. For example, subjects afflicted with lupus or multiple sclerosis are amenable to treatment with the methods of the present invention.

A further understanding of the nature and advantages of the present invention may be realized by reference to the remaining portions of the specification and claims.

### DESCRIPTION OF THE DRAWINGS

FIGS. 1A-1B show lymphoaccumulation and T cell phenotype in MRL-Fas^{lpr} mice. (A) T cell accumulation. LN cells were obtained from MRL-Fas^{lpr} and control C57BL/6 mice at the indicated ages. Frequency and number of CD4⁺, CD8⁺ and DN T cells were determined by flow cytometry after gating on the TCRβ⁺ cell population. (B) T cell phenotype. Expression of CD44, CD69, CD25 and CD62L by TCRβ⁺CD4⁺, TCRβ⁺CD8⁺, and TCRβ⁺DN T cells was assessed by flow cytometry in young (Y, 8 weeks of age) and older (0, 12 weeks of age) MRL-Fas^{lpr} mice. Data are representative of 3-10 independent experiments with 3-5 mice/group.

FIGS. 2A-2C show cytokine receptor expression and survival of MRL-Fas^{lpr} T cells. (A) CD127 and CD122 down-regulation in DN T cells. Lymph node T cells from young (8 weeks of age) and older (12 weeks) MRL-Fas^{lpr} mice were analyzed for expression of IL-7Rα (CD127), IL-15Rβ (CD122), γc (CD132) and IL-21R. Similar profiles were obtained with spleen cells. (B) Effect of IL-7 on T cell survival. Aliquots (5×10⁶) of LN cells were cultured with or without IL-7 (20 ng/ml), and numbers of viable CD4⁺, CD8⁺ and DN T cells were determined at the indicated time-points. Data are representative of three separate experiments. (C) Effect of IL-21 on DN T cell survival. Purified DN T cells (5×10⁶) were cultured for 3 days in medium (control, C), or in the presence of IL-7 (20 ng/ml), IL-15 (10 ng/ml) or IL-21 (50 ng/ml, top panel; 50-200 ng/ml, middle panel). In addition, DN T cells were CFSE-labeled, cultured with or without IL-21 (200 ng/ml), and proliferation measured by flow cytometry (lower panel). Data are representative of 2-3 independent experiments with 2-5 mice/group. Asterisks indicate statistical significance (p<0.05).

FIGS. 3A-3D show MRL-Fas^{lpr} T cell proliferation and conversion to DN T cells in T cell-deficient recipients. (A) Homeostatic and spontaneous proliferation. Purified CD4⁺, CD8⁺, and DN T cells from 12 week-old MRL-Fas^{lpr} mice were labeled with CFSE and 5×10⁶ cells transfused to TCRβ^{-/-} recipients. After 7 days, TCRβ⁺ T cell subsets from peripheral LNs were analyzed by flow cytometry. (B) Kinetics of homeostatic and spontaneous proliferation. Frequencies of slow-proliferating (1-6 divisions) and fast-proliferating (>7 divisions) cells at the indicated time points are depicted. (C) Conversion of CD8⁺ T cells to DN T cells during spontaneous proliferation. Purified CD4⁺ and CD8⁺ T cells were CFSE-labeled and transfused to TCRβ^{-/-} hosts. Gated TCRβ⁺CD8⁻ (left panel) and TCRβ⁺CD4⁻ (right panel) cells were analyzed 1, 2 or 3 weeks post-transfer for the expression of the CD4 and CD8 coreceptors, respectively. (D) Conversion to DN T cells during spontaneous proliferation is accompanied by down-regulation of CD127 and CD122. Purified CD8⁺ T cells were CFSE-labeled, transfused into TCRβ^{-/-} hosts, and 12 days post-transfer, gated TCRβ⁺CD4⁻ cells were analyzed for the expression of CD8, CD127 and CD122. Data are representative of 2-3 independent experiments with 3-4 mice/group.

FIGS. 4A-4E show decreased consumption and increased production of IL-7 in MRL-Fas^{lpr} mice. (A) DN T cells do not inhibit homeostatic T cell proliferation. CFSE-labeled T cells (3×10⁶) from young (8 weeks) MRL-Fas^{lpr} mice were transfused into TCRβ^{-/-}MRL-Fas^{lpr} recipients alone (control) or together with 50-100×10⁶ of either total LN T cells (mostly naïve, lacking DN cells) from young (6 weeks) mice (left panel), or purified DN T cells from older (12 weeks) mice (right panel). Data represent percentages (average±SD) of cells in various cell divisions as determined by CFSE profiles. (B) T cell proliferation in non-lymphopenic hosts. CFSE-labeled LN T cells (20×10⁶) from older (16 weeks) MRL-Fas^{lpr} mice were transferred into age-matched unmanipulated (non-lymphopenic) wild-type syngeneic recipients. As a control, LN T cells from non-autoimmune C57BL/6 (B6) mice were transferred into syngeneic recipients. Seven or 15 days after transfer, LN and spleen cells were harvested and donor CFSE⁺ T cells detected by gating the TCRβ⁺ cell populations. The frequency of donor cells in divisions 0 to 4 was calculated as a percentage of CFSE⁺ cells. To examine the effect of IL-7 blockade, groups of transfused MRL-Fas^{lpr} were treated with PBS or anti-CD127 antibody (200 μg i.p. 3 times/week). (C) Transcript level analysis. RT-PCR was performed to examine IL-7 and CD 127 transcript levels in LNs of young (Y, 6 weeks) and older (0, 20 weeks) MRL-Fas^{lpr} and control C57BL/6 (B6) mice. Data were normalized using HPRT1 and expressed as fold change compared to transcript levels in B6 LNs (arbitrarily set to 1). (D) Flow cytometry analysis of fibroblastic reticular cells (FRCs). LN cells of MRL-Fas^{lpr} and control B6 mice were stained with antibodies to CD45, gp38 (podoplanin) and CD31. Representative plots are shown for gated CD45-negative cells. The frequency of FRCs (CD31⁻ gp38⁺) in the CD45⁻ LN cell population is indicated. (E) Accumulation of FRCs and CD127⁺ T cells in MRL-Fas^{lpr} mice. The number of FRCs and CD127⁺ T cells in young and older MRL-Fas^{lpr} compared to B6 controls was determined by flow cytometry. Data are representative of 2-3 independent experiments with 3-5 mice/group. Asterisks indicate statistical significance (p<0.05).

FIGS. 5A-5F show that IL-7R blockade inhibits disease in MRL-Fas^{lpr} mice. (a) IL-7R blockade by anti-CD127 antibody. MRL-Fas^{lpr} mice (6 weeks old) were treated i.p. with 200 μg of anti-CD127 antibody (A7R34) or PBS, 3 times weekly for 4 weeks, and expression of CD127 was determined. (b-d) Effects of prophylactic anti-CD127 antibody treatment. Young female MRL-Fas^{lpr} mice (6 weeks old) were treated with anti-CD127 or PBS for 6 to 10 weeks. Antibody levels (c) were assessed after 6 weeks, dermatitis (b) after 8 weeks, and weights of inguinal, axillary and cervical LNs and spleen (d) after 10 weeks of treatment. (e-f) Effects of therapeutic anti-CD 127 antibody treatment. Female MRL-Fas^{lpr} mice (14 weeks old) were treated with anti-CD127 or PBS for 3 to 10 weeks. Proteinuria, glomerulonephritis (GN) and lymphocytic infiltration (LI) were determined between 14 and 20 weeks of age (e), and survival at 24 weeks of age (f). Data are representative of 1-5 independent experiments with 3-9 mice/group.

FIG. 6 shows CD25 expression in MRL-Fas^{lpr} T cells. LN cells from C57BL/6 (12 weeks of age) and MRL-Fas^{lpr} (8 or 16 weeks) mice were stained with antibodies to TCRβ, CD4, CD25 and intracellular Foxp3 and examined by flow cytometry. Representative results for gated TCRβ⁺CD4⁺ T cells are depicted. Data are representative of 2 independent experiments with 3-5 mice/group.

FIG. 7 shows permanent CD127 down-regulation in DN T cells. LN cells (5×10⁶) from MRL-Fas^{lpr} mice (20 weeks of age) were analyzed either ex vivo, or after 24 hr in vitro culture in the presence of absence of IL-7 (20 ng/ml). Data are representative of 2 independent experiments with 2 mice/group examined in triplicate cultures.

FIG. 8 shows reduced spontaneous proliferation of CD8⁺ T cells in chronically immunodeficient TCRβ^{-/-} MRL-Fas^{lpr} recipients. (a) CD4⁺, CD8⁺, and DN T cells from 12 week-old MRL-Fas^{lpr} mice were purified by flow cytometry, labeled with CFSE, and 5×10⁶ of each cell subset transfused to TCRβ^{-/-} recipients. CFSE profiles were assessed in peripheral (cervical, brachial, inguinal) LNs 14 days post-transfer. (b) Total LN cells from MRL-Fas^{lpr} mice were labeled with CFSE, transferred to TCRβ^{-/-} recipients, and CFSE profiles examined 13 days post-transfer in peripheral LNs, mesenteric LNs and spleen. As controls, LN cells from C57BL/6 mice were CFSE-labeled and transferred to RAG2^{-/-} C57BL/6 recipients. Data are representative of 1-5 independent experiments with 3 mice/group.

FIG. 9 shows reduced spontaneous proliferation of CD8⁺ T cells in sublethally irradiated TCRβ^{-/-} MRL-Fas^{lpr} recipients. TCRβ^{-/-} MRL-Fas^{lpr} were sublethally irradiated (600 rad) or not, and 24 hr later transfused with CFSE-labeled total LN cells from MRL-Fas^{lpr} mice. CFSE profiles were examined 7 days post-transfer in peripheral LNs. Data are from 1 experiment with 3 mice/group.

FIG. 10 shows accumulation of FRCs and CD127⁺ T cells in LNs of MRL-Fas^{lpr} mice. LN cells were isolated from mice displaying various levels of lymphadenopathy. The numbers of FRCs (CD45⁻ CD31⁻ gp38⁺) and CD127⁺ T cells (TCRβ⁺) were determined by flow cytometry and plotted as a function of the number of total LN cells for each individual mouse. Linear regression and goodness of fit (r) were calculated using Prism 4 software. Dotted lines indicate the frequency±1 STD of FRCs (left panel) and CD127⁺ T cells (right panel) as determined in young mice with no lymphadenopathy, and predict how these cell types would accumulate if their frequencies were maintained at a constant. Data are a combination of 2 independent experiments with 3 mice/group.

FIG. 11 shows that anti-CD 127 treatment inhibits IL-7-mediated STAT5 phosphorylation. Splenocytes (5×10⁶) were cultured for 10 min with recombinant IL-7 (100 ng/ml), combinations of IL-7 and anti-CD127 antibody (A7R34, 10 μg/ml), or medium alone (filled profiles). Cells were then stained for CD4 and intracellular pSTAT5, and analyzed by flow cytometry. Data are representative of 2 independent experiments with 2 mice/group analyzed in triplicate.

FIGS. 12A-12C show that IL-7R blockade inhibits T and B cell accumulation in MRL-Fas^{lpr} mice. Young (6 weeks) MRL-Fas^{lpr} mice were treated i.p. with 200 μg anti-CD127 antibodies or PBS, 3 times weekly for 4 weeks. (a) Total T (TCRβ⁺) and B (CD19⁺) cell numbers in LNs and spleen. (b) Peripheral T cell subsets. (c) Peripheral B cell subsets. Data are representative of 2-3 independent experiments with 4 mice/group. Asterisks indicate statistical significance (p<0.05).

FIGS. 13A-13B show effect of IL-7R blockade on B and T cell subsets. Young (6 weeks) MRL-Fas^{lpr} mice were treated i.p. with 200 μg anti-CD127 antibodies or PBS, 3 times weekly for 4 weeks. (a) B cell subsets in bone marrow (pre-B and pro-B cells, CD19^{hi}Igk⁻; newly formed B cells, CD19^{int}Igk⁺; recirculating B cells, CD19^{hi}Igk⁺), spleen (immature, T1; follicular, T2-F0; marginal zone, MZ), and peritoneal cavity (CD5^{low}B220^{low}). (b) Thymocyte subsets. Double negative (DN, CD4⁻CD8⁻) thymocytes were subdivided into DN I (CD44⁺CD25⁻), DN II (CD44⁺CD25⁺), DN III (CD44⁻CD25⁺) and DN IV (CD44⁻CD25⁻) subsets, while double-positive (DP, CD4⁺CD8⁺) and single-positive (SP, CD4⁺CD8⁻ and CD4⁻CD8⁺) thymocytes were subdivided into TCRβ⁻, TCRβ^{low}, TCRβ^{int}, and TCRβ^{hi} subsets. Significant (*, p<0.05) changes in anti-CD127-treated vs. control mice were found in the bone marrow for pre-/pro-B cells (5.9±0.5% vs. 24.4±1.7%), newly formed B cells (2.1±0.4% vs. 4.5±0.1%) and recirculating B cells (1.6±0.2% vs. 3.8±0.3%), and in the thymus for DN II cells (3.2±0.3% vs. 6.2±0.1%), TCRβ⁻ cells (18.6±9.3% vs. 13.5±0.7%), TCRβ^{int} cells (6.2±0.4% vs. 7.6±0.4%), and TCRβ^{hi} cells (6.3±0.8% vs. 9.8±0.2%). Data are representative of 2 independent experiments with 4 mice/group.

FIGS. 14A-14G show that IL-7 is required for CD4⁺ T cell activation. (A) Anti-IL-7RA antibodies block PLP-peptide induced upregulation of the cell-surface activation markers CD69 and CD25 on PLP-TCR transgenic CD4⁺ T cells. Shown are histograms of CD69 or CD25 cell-surface levels at 1, 2 or 7 days post-treatment of the indicated stimuli and antibodies. (B) IL-7R blockade inhibits TCR-induced Ca²⁺-mobilization in CD4⁺ T cells. CD4⁺ T cells were cultured with anti-IL-7RA (blue) or isotype control (red) antibodies for 1 hr, loaded with Indo-1 AM, stimulated with Ionomycin (1 μM) or anti-CD3/CD28 antibodies, and ratiometric analysis of intracellular Ca²⁺ levels of gated CD4⁺CD44^{lo} cells determined. (C) Reduced PLP-specific CD4⁺ TCR transgenic T cell proliferation to PLP in the presence of anti-IL-7RA antibodies and APCs. Proliferating cells were identified by FACS via CFSE-dilution, denoted by black bars. (D) The presence of anti-IL-7Rα (blue), but not control (red), antibodies causes increased apoptosis of PLP-TCR transgenic CD4⁺ T cells 48 hrs following PLP stimulation. (E) The presence of anti-IL-7Rα (blue), but not control (red), antibodies blocks the accumulation of Bcl-2 in PLP-TCR transgenic CD4⁺ T cells stimulated with PLP for up to 4 days. Day 4 is depicted. (F) Transgenic Bcl-2-expression rescues CD4⁺ T cells from IL-7R blockade-induced AICD, but does not restore the activation defect. Wild-type or Bcl-2 transgenic C57BL/6 splenocytes were pretreated with anti-IL-7Rα or isotype antibodies and then stimulated with anti-CD3/28 antibodies. Annexin V, CD25, and CD69 expression was analyzed on CD4⁺ T cells every 24 hrs for 96 hrs. 24 and 48 hour time points are depicted. Numbers indicate % cells in the respective gate. (G) The Bcl-2 transgene fails to rescue TCR-induced CD4⁺ T cell proliferation in the presence of anti-IL-7Rα antibodies. For panels A, C, D, and E, splenocytes from PLP-TCR transgenic mice were pretreated with anti-IL-7Rα or isotype control antibodies (10-100 μg/ml each), cultured with or without IL-7 (10 ng/ml) as indicated, and stimulated with PLP (10 μg/ml). All FACS plots are gated on CD4⁺ T cells. The data are representative of 3-6 independent experiments.

FIGS. 15A-15F show that IL-7 via STAT5 and PI3K/Akt phosphorylation is distinctly required for CD4⁺ T cell activation. (A) TCR-stimulation does not activate CD4⁺ T cells from IL-7^{-/-} mice, but induces apoptosis in a manner that is rescued by rIL-7. Splenocytes from wild-type (red) or IL-7^{-/-} (blue) mice were activated with anti-CD3/CD28 antibodies for 48 hrs and CD69 and CD25 cell-surface levels were determined by on CD4⁺ T cells by FACS. Addition of rIL-7 (10 ng/ml, orange) prevented apoptosis (Annexin V upregulation) of IL-7^{-/-} CD4⁺ T cells and partially restored activation. Green lines, unstimulated IL-7^{-/-} CD4⁺ T cells. (B) CD4⁺ T cells from IL-7^{-/-} mice mobilize Ca²⁺ after ionomycin, but not anti-CD3/CD28 stimulation. (C) Wild-type splenocytes restore TCR-activation of CD4⁺ T cells from IL-7^{-/-} mice in mixed cultures. Splenocytes from Ly5a⁻IL-7^{-/-} and wild-type Ly5a⁺ mice were mixed 1:1 and cultured for the indicated time points with anti-CD3/CD28 antibodies. CD69 and CD25 expression on gated Ly5a⁻IL-7^{-/-} (blue) and wild-type Ly5a⁺ CD4⁺ T cells (red) was determined by FACS. Green lines, unstimulated IL-7^{-/-} CD4⁺ T cells. (D) IL-7 signaling is partially required for CD8⁺ T cell activation. Wild-type (red) and IL-7^{-/-} (blue) CD8⁺ T cells were activated with anti-CD3/CD28 and CD69 and CD25 expression determined by FACS. Green, unstimulated IL-7^{-/-} CD8⁺ T cells. (E) PLP-TCR transgenic CD4⁺ T cells show a stress phenotype in the presence of anti-IL-7Rα antibodies. Splenocytes from PLP-TCR transgenic mice were pretreated with anti-IL-7Rα or isotype antibodies, then stimulated with PLP. Time points up to 2 hrs were analyzed by FACS for phosphorylation (activation) of Akt, p38, NF-κB p65, INK, and Erk1/2. Histograms depict the 20 min time point of maximal activation in isotype-treated controls. Blue, IL-7Rα antibody-treated CD4⁺ T cells; red, isotype-treated CD4⁺ T cells; green, unstimulated CD4⁺ T cells. (F) TCR stimulation induces STAT5 phosphorylation in an IL-7-dependent manner. Splenocytes from PLP-TCR transgenic mice were pretreated with anti-IL-7Rα or isotype antibodies and then stimulated with PLP. CD4⁺ T cells were analyzed at the indicated time points by FACS for STAT5 phosphorylation (activation). Blue, IL-7Rα antibody-treated cells; red, isotype-treated cells. All data are representative of 3-5 independent experiments.

FIGS. 16A-16D show that engaged PLP-specific TCR transgenic CD4⁺ T cells are preferentially eliminated following anti-IL-7Rα antibody administration compared to naive host non-transgenic CD4⁺ T cells. (A) Undivided (CFSE^{hi}) PLP-specific CD4⁺ TCR transgenic T cells (indicated by bar) are mostly eliminated from LN following a short course of anti-IL-7Rα antibody treatment (blue line) compared to isotype-treated controls (red line). n=4-5 mice/group; p<0.001. (B) Increased in vivo apoptosis of undivided (CFSE^{hi}) CD4⁺ TCR transgenic CD4⁺ T cells obtained from anti-IL-7Rα antibody-treated mice in A (indicated by bar) compared to controls. n=4-5 mice/group; p<0.001. (C) No loss of PLP-specific TCR transgenic CD4⁺ T cells in unimmunized SJL recipients treated with anti-IL-7RA or isotype control antibodies for 3 days. (D) After transfer, fewer PLP-specific CD4⁺ TCR transgenic T cells were recovered from spleens of anti-IL-7RA antibody-treated recipient mice compared to controls. n=3-4 mice/group; p<0.01. Data are representative of 2-3 independent experiments.

FIGS. 17A-17I show that R-EAE is attenuated by anti-IL-7RA antibodies. (A) Incidence and (B) severity of R-EAE is markedly reduced by administration of anti-IL-7RA (open circles) compared to isotype control antibodies (closed circles). n=8-11 mice/group; p<0.001. (C) Increased conversion of procaspases 3 and 8 to active caspases in CD4⁺ T cells obtained two weeks after R-EAE induction from anti-IL-7RA-antibody treated mice (blue line) compared to controls (red line). n=8-11 mice/group; p<0.01. Reduced (D) incidence and (E) severity of polarized T_{H}1-induced adoptively transferred R-EAE following anti-IL-7RA antibody (open circles) compared to isotype antibody (closed circles) treatment. n=5-8 mice/group; p<0.001. (F) Incidence and (G) severity of R-EAE is reduced if anti-IL-7RA antibodies are administered at the start of the acute disease phase (day 8). n=8-11 mice/group; p<0.001 from day 8 until end. (H) Incidence and (I) severity of R-EAE associated relapses are reduced when anti-IL-7RA antibody treatment is initiated prior to the first relapse (day 20). n=8-11 mice/group; p<0.001 from day 20 until end. (A, B, F, G, H, and I) Young female SJL mice were injected with PLP peptide (emulsified in CFA) plus PTX to induce R-EAE or (D and E) CD4⁺ T cells were obtained from PLP immunized SJL donors, polarized in vitro with PLP and IL-12 to a T_{H}1-type cell (24) then transferred to naïve syngeneic recipients. Arrows indicate initiation of anti-IL-7RA or isotype control antibody treatment by i.p. injection. Data are expressed as either daily group means (incidence) or mean±SEM (EAE Severity Score), and are representative of 3-5 independent experiments.

FIG. 18 shows that IL-7Rα (CD127) is downregulated on CD4⁺ T cells in vivo following antigen engagement. R-EAE was induced in Thy1.2 SJL mice and on day 10 naive Thy1.1 PLP-specific CD4⁺ TCR transgenic T cells (7×10⁶ cells/mouse) were transferred. Transgenic CD4⁺ T cells recovered from spleens and LNs were analyzed for expression of CD 127 at the indicated time points.

FIG. 19 shows that IL-7Rα (CD127) is downregulated on CD4⁺ T cells in vitro following activation by peptide or IL-7. Splenocytes from PLP-specific TCR transgenic mice were stimulated with PLP (10 μg/ml) or IL-7 (10 ng/ml) for the indicated time points and CD127 expression on CD4⁺ T cells was determined by FACS. Blue lines indicate treated cells, red lines untreated cells.

FIGS. 20A-20B show that IL-7Rα blockade reduces antigen-induced proliferation and cytokine production. (A) Decreased proliferation to PLP from anti-IL-7Rα-antibody-treated (open bars) compared to isotype antibody-treated mice induced for R-EAE (solid bars). n=8-11 mice/group. (B) Reduced production of proinflammatory cytokines in response to PLP stimulation by anti-IL-7Rα-antibody-treated CD4⁺ T cells (open bars) compared to controls (solid bars). n=8-11 mice/group. In both panels, EAE was induced in anti-IL-7Rα or isotype control antibody-treated Sit mice, splenocytes restimulated with PLP on day 14 post EAE-induction, and proliferation and cytokine production measured by ³H-thymidine incorporation or ELISA, respectively. * indicates p<0.05.

### DETAILED DESCRIPTION OF THE INVENTION

### I. Overview

The present invention is predicated in part on the discovery by the present inventors that hyperactive immune responses (e.g., as manifested in systemic autoimmunity) are associated with excess IL-7 and inhibited by IL-7R blockade. The inventors performed studies to define homeostatic characteristics of T cells in the MRL-Fas^{lpr} lupus model, focusing on potential imbalances in production and consumption of the major T cell prosurvival cytokines. Specifically, the inventors discovered that increased availability of IL-7, caused by persistent down-regulation of the IL-7R in chronically activated T cells (overactivated T cells) and expansion of stromal cells that produce this cytokine, contributes to lupus-like disease in the MRL-Fas^{lpr} model. It was found that a large proportion of T cells displayed phenotypic markers resembling those of T cells undergoing homeostatic proliferation. The CD4⁻CD8⁻ double-negative (DN) T cells, which are associated with autoimmunity, massively accumulate in this Fas-defective lupus model. The inventors observed that these DN T cells do not express receptors for IL-7 and IL-15, leading to reduced consumption of these cytokines. IL-15 was also found to enhance the in vitro survival of CD8+ precursor T cells. Moreover, lymphoaccumulation in the MRL-Fas^{lpr} mice was found to be associated with expansion of FRCs, increased IL-7 production, and enhanced IL-7-dependent homeostatic T cell proliferation. Importantly, it was further observed that treatment with an IL-7RA-blocking antibody significantly reduced autoimmune disease manifestations in this model.

The present inventors additionally discovered that IL-7 is integral for efficient activation of CD4⁺ T cells. This novel finding adds IL-7 as an essential “third signal” to the current paradigm that T cell activation requires only the combined stimulation of TCR and costimulatory receptors. The findings uncover a novel function for IL-7 that is essential for CD4⁺ T cell activation which is distinct from its established role in promoting T cell survival. It was found that, contrary to the paradigm that early TCR-induced T cell activation is cytokine-independent, IL-7 is required at the time of antigen engagement or shortly thereafter. This novel costimulatory function of IL-7 promotes CD4⁺ T cell activation by providing key components necessary for efficient TCR-mediated activation, such as activated STAT5 and PI3K/Akt. It was further found that antibody-mediated blockade of IL-7Rα can not only avert the development of experimental autoimmune encephalomyelitis (EAE), the recognized mouse model of multiple sclerosis (MS), but also resolve relapses. Strikingly and of clinical relevance, short-term IL-7Rα blockade selectively depleted recently activated autoantigen-specific CD4⁺ T cells, but left the non-engaged broad T cell repertoire largely unaffected. These findings indicate that, beyond MS, short-term IL-7a blockade have wider therapeutic applicability to other autoimmune/inflammatory syndromes mediated by helper CD4⁺ T cells.

In accordance with these discoveries, the present invention provides methods for treating subjects suffering from disorders associated with hyperactive immune responses. The invention also provides methods for inhibiting proliferation of overactivated T cells (e.g., CD4⁻CD8⁻ T cells) and methods for inhibiting activation of CD4⁺ T cells. Related pharmaceutical compositions and kits are also provided in the invention.

The following sections provide a more detailed guidance for practicing the invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “analog” or “derivative” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs to identify variants of known compounds having improved traits (such as higher binding affinity for a target molecule) is an approach that is well known in pharmaceutical chemistry.

Administration “in conjunction with” one or more other therapeutic agents includes simultaneous (concurrent) and consecutive administration in any order.

“Autoimmune disease” refers to a disease caused by an inability of the immune system to distinguish foreign molecules from self molecules, and a loss of immunological tolerance to self antigens, which results in destruction of the self molecules. Examples of autoimmune diseases include but are not limited to systemic lupus erythematosus, Sjogren's syndrome, scleroderma, ulcerative colitis, insulin-dependent diabetes mellitus (IDDM), multiple sclerosis, and rheumatoid arthritis.

“Autoantigen” refers to a self-antigen normally found within a mammal and normally recognized as self, but due to an auto-immune disease, is erroneously recognized as foreign by the mammal. That is, an autoantigen is not recognized as part of the mammal itself by the lymphocytes or antibodies of that mammal and is erroneously attacked by the immunoregulatory system of the mammal as though such autoantigen were a foreign substance. An autoantigen according to the invention also includes an epitope or a combination of epitopes derived from that autoantigen.

Overactivation of a T cell refers to excessive or uncontrolled proliferation of a T cell (e.g., CD4⁺ T cell, CD8⁺ T cell or CD4⁻CD8⁻ T cell) and/or its precursors. It is often associated with overproduction of cytokines by these cells. In the context of the present invention, an overactivated T cell is also characterized by the downregulation of IL-7 receptor on the cells and the increased level of IL-7.

The term “contacting” has its normal meaning and refers to combining two or more agents (e.g., polypeptides or small molecule compounds) or combining agents and cells. Contacting can occur in vitro, e.g., combining two or more agents or combining an agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate. Contacting can also occur inside the body of a subject, e.g., by administering to the subject an agent which then interacts with the intended target (e.g., a tissue or a cell).

Disorders associated with a hyperactive immune system or hyperactive immune responses refer to diseases or conditions that involve or are mediated by an overactive immune system which attacks the body's own tissues. Examples of these disorders include rheumatoid arthritis, type I diabetes, multiple sclerosis, lupus, neuromyelitis optica (NMO), Sjogren's syndrome, Crohn's disease (intestine destruction) and others. The term as used herein also broadly encompasses other conditions associated undesired immune responses, e.g., transplantation rejection.

“Inflammation” or “inflammatory response” refers to an innate immune response that occurs when tissues are injured by bacteria, trauma, toxins, heat, or any other cause. The damaged tissue releases compounds including histamine, bradykinin, and serotonin. Inflammation refers to both acute responses (i.e., responses in which the inflammatory processes are active) and chronic responses (i.e., responses marked by slow progression and formation of new connective tissue). Acute and chronic inflammation can be distinguished by the cell types involved. Acute inflammation often involves polymorphonuclear neutrophils; whereas chronic inflammation is normally characterized by a lymphohistiocytic and/or granulomatous response. Inflammation includes reactions of both the specific and non-specific defense systems. A specific defense system reaction is a specific immune system reaction response to an antigen (possibly including an autoantigen). A non-specific defense system reaction is an inflammatory response mediated by leukocytes incapable of immunological memory. Such cells include granulocytes, macrophages, neutrophils and eosinophils.

Systemic lupus erythematosus (SLE or lupus) is a chronic autoimmune connective tissue disease that can affect any part of the body. As occurs in other autoimmune diseases, the immune system attacks the body's cells and tissue, resulting in inflammation and tissue damage. SLE most often harms the heart, joints, skin, lungs, blood vessels, liver, kidneys, and nervous system. The course of the disease is unpredictable, with periods of illness (called flares) alternating with remissions. The disease occurs nine times more often in women than in men, especially between the ages of 15 and 50, and is more common in those of non-European descent. SLE is often treated by addressing its symptoms, mainly with cyclophosphamides, corticosteroids and immunosuppressants, and there is currently no cure.

Multiple sclerosis (MS) is a disease in which the fatty myelin sheaths around the axons of the brain and spinal cord are damaged, leading to demyelination and scarring as well as a broad spectrum of signs and symptoms. Disease onset usually occurs in young adults, and it is more common in females. MS affects the ability of nerve cells in the brain and spinal cord to communicate with each other. Nerve cells communicate by sending electrical signals called action potentials down long fibers called axons, which are wrapped in an insulating substance called myelin. In MS, the body's own immune system attacks and damages the myelin. When myelin is lost, the axons can no longer effectively conduct signals. The name multiple sclerosis refers to scars (scleroses—better known as plaques or lesions) in the white matter of the brain and spinal cord, which is mainly composed of myelin. Almost any neurological symptom can appear with the disease, and often progresses to physical and cognitive disability. MS takes several forms, with new symptoms occurring either in discrete attacks (relapsing forms) or slowly accumulating over time (progressive forms). Between attacks, symptoms may go away completely, but permanent neurological problems often occur, especially as the disease advances. There is no known cure for MS. Existing treatments attempt to return function after an attack, prevent new attacks, and prevent disability.

T lymphocytes recognize antigens as peptides bound to major histocompatibility complex (MHC) molecules. The fine specificity of the T cell is defined by the T-cell receptor (TCR). Most T lymphocytes express the TCR αβ and either CD4 or CD8 molecules. These molecules stabilize the TCR-peptide/MHC interaction, are essential for intrathymic selection, and contribute to transmembrane signaling, with important roles in the development and activation of helper and cytotoxic T cells. All T cells originate from haematopoietic stem cells in the bone marrow. Haematopoietic progenitors derived from haematopoietic stem cells populate the thymus and expand by cell division to generate a large population of immature thymocytes. The earliest thymocytes express neither CD4 nor CD8. As they progress through their development they become double-positive thymocytes (CD4⁺CD8⁺), and finally mature to single-positive (CD4⁺CD8⁻ or CD4⁻CD8⁺) thymocytes that are then released from the thymus to peripheral tissues. These precursor cells then different into various type of mature or effector T cells which can be CD4⁺ T cells, CD8⁺ T cells, or CD4⁻CD8⁻ double negative (DN) T cells.

T helper (Th) cells assist other white blood cells in immunologic processes, including maturation of B cells into plasma cells and activation of cytotoxic T cells and macrophages, among other functions. These cells are also known as CD4⁺ T cells because they express the CD4 protein on their surface. Helper T cells become activated when they are presented with peptide antigens by MHC class II molecules that are expressed on the surface of Antigen Presenting Cells (APCs). Once activated, they divide rapidly and secrete small proteins called cytokines that regulate or assist in the active immune response. These cells can differentiate into one of several subtypes, including TH1, TH2, TH3, TH17, or TFH, which secrete different cytokines to facilitate a different type of immune response. The mechanism by which T cells are directed into a particular subtype is poorly understood, though signaling patterns from the APC are thought to play an important role.

Cytotoxic T cells (TC cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I, which is present on the surface of nearly every cell of the body. Through IL-10, adenosine and other molecules secreted by regulatory T cells, the CD8⁺ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise two subtypes: central memory T cells (TCM cells) and effector memory T cells (TEM cells). Memory cells may be either CD4⁺ or CD8⁺. Memory T cells typically express the cell surface protein CD45RO.

Other than CD4⁺ or CD8⁺ T cells, a small population of all T cells lacking these coreceptors. These T cells, CD4⁻ CD8⁻ (double negative (DN) T cells), have been identified in the peripheral immune system of mice and humans and have been associated with human and murine autoimmune and immunodeficiency diseases. While little is known concerning DN T cells and their direct role in disease pathology or normal immunity in humans, recent studies indicate that the lack of coreceptors allows this population to tolerate chronic stimulation. In contrast, chronic stimulation of CD4⁺ or CD8⁺ T cells limits their expansion through an apoptosis-dependent mechanism. In the murine model, αβ DN T cells exhibit markers of activation/memory, a lowered threshold of activation, ex vivo cytolytic activity, and the ability to rapidly secrete gamma interferon (IFN-γ). In healthy humans, DN αβ T cells have been identified in peripheral blood, thymus, and skin.

Small interfering RNA (siRNA), sometimes known as short interfering RNA or silencing RNA, have a well-defined structure: a short (20-25 nt, e.g., 21 nt) double strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells by various transfection methods to bring about the specific knockdown of a gene of interest. Essentially any gene of which the sequence is known can thus be targeted based on sequence complementarity with an appropriately tailored siRNA. Transfection of an exogenous siRNA can be problematic because the gene knockdown effect is only transient, particularly in rapidly dividing cells. One way of overcoming this challenge is to modify the siRNA in such a way as to allow it to be expressed by an appropriate vector, e.g., a plasmid. This is done by the introduction of a loop between the two strands, thus producing a single transcript, which can be processed into a functional siRNA. Such transcription cassettes typically use an RNA polymerase III promoter (e.g., U6 or H1), which usually directs the transcription of small nuclear RNAs (snRNAs) (U6 is involved in gene splicing; H1 is the RNase component of human RNase P). The resulting siRNA transcript is believed to be processed subsequently by Dicer.

A small hairpin RNA or short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it.

The term “subject” for purposes of treatment refers to any animal classified as a mammal, e.g., human and non-human mammals. Examples of non-human animals include dogs, cats, cattle, horses, sheep, pigs, goats, rabbits, and etc. Unless otherwise noted, the terms “patient” or “subject” are used herein interchangeably. Preferably, the subject is human.

The term “substantially identical” or “substantial identity” when referring to nucleic acid or amino acid sequences means that a nucleic acid or amino acid sequence comprises a sequence that has at least 90% sequence identity or more, preferably at least 95%, more preferably at least 98% and most preferably at least 99%, with a reference sequence as measured with one of the commonly used bioinformatics programs (preferably BLAST). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

The term “treating” or “alleviating” includes the administration of compounds or agents to a subject to prevent or delay the onset of the symptoms, complications, or biochemical indicia of a disease (e.g., an inflammatory disorder or an autoimmune disease), alleviating the symptoms or arresting or inhibiting further development of the disease, condition, or disorder. Subjects in need of treatment include those already suffering from the disease or disorder as well as those being at risk of developing the disorder. Treatment may be prophylactic (to prevent or delay the onset of the disease, or to prevent the manifestation of clinical or subclinical symptoms thereof) or therapeutic suppression or alleviation of symptoms after the manifestation of the disease. In the treatment of a disease or disorder associated with or mediated by systemic autoimmunity or neuroinflammation, a therapeutic agent may directly decrease the pathology of the disease, or render the disease more susceptible to treatment by other therapeutic agents.

### III. IL-7 and IL-15 Signaling and Antagonist Compounds

The methods of present invention are directed to suppressing IL-7 or IL-15 expression or signaling in order to inhibit activation or proliferation of CD4⁺ T cells or CD4⁻ CD8⁻ DN T cells. This is achieved by the employment of an antagonist compound that specifically down-regulates or suppresses expression of the target molecule (e.g., IL-7, IL-15 or their receptor) or antagonizes a cellular activity mediated by the target molecule (e.g., binding by IL-7 and L-15 to their respective receptor). As detailed below, such antagonist compounds include, e.g., antibodies that specifically recognize IL-7R or IL-15R and inhibitory polynucleotides that downregulate expression or cellular level of the cytokine or its receptor. Other agents that antagonize IL-7 or IL15 signaling, e.g., compounds which inhibit IL-7 or IL-15 signaling (e.g., small organic compounds, peptides or mimetics) can also be used in the practice of the invention.

The target cytokines (IL-7 and IL-15) and their receptors from various species can be used in the preparation of antagonist compounds disclosed herein. For targeting human cells or human subjects, the antagonists employed in the invention are preferably designed and prepared based on structural information (e.g., amino acid sequence or nucleotide sequence) of human IL-7 (or IL-15) and human IL-7R (or IL-15R). However, due to the substantial sequence identity and cross-reactivity among the different cytokine orthologs, antagonist compounds for a cytokine and its receptor from other species (e.g., rat or mouse) can also be readily utilized in the practice of the present invention. For example, human IL-7 and mouse IL-7 share at the amino acid sequence level approximately 60% identity. More importantly, human and mouse IL-7 exhibit no species-specificity. For example, human IL-7 is equally active on both human and mouse cells, mouse IL-7 is also active on human T cells.

Similarly, substantial sequence homology exists among different IL-7R orthologs, e.g., between human IL-7R and mouse IL-7R. The biological effects of IL-7 are initiated by binding of the cytokine to a receptor complex consisting of a ligand specific binding component, IL-7Rα, and a second component, the γc (gamma common) chain. The γc chain is also a part of the receptor complexes associated with IL-2, IL-4, IL-9, and IL-15 binding and signal transduction. The human IL-7Rα is produced as a 459 aa residue precursor with a 20 aa residue signal peptide, a 25 aa residue transmembrane domain, an extracellular domain of 219 aa residues, and a cytoplasmic domain of 195 aa residues. The cDNA for the mature protein predicts a size of 49.5 kDa, but the observed size of the receptor is approximately 75 kDa, presumably as a result of post-translational glycosylation. The mouse IL-7Rα is the same size and shows approximately 64% sequence identity with the human protein at the amino acid sequence level.

IL-7 and IL15 cytokines, as well as their receptors, from many species are known to the skilled artisans. Their sequences and functional characterization have been reported in the art. For example, human IL-7 sequence was described in Goodwin et al., Proc. Natl. Acad. Sci. U.S.A. 86:302-306, 1989. Mouse IL-7 gene was characterized in Lupton et al., J. Immunol. 144:3592-3601, 1990. Human and mouse IL-7 receptor sequences were reported in Goodwin et al., Cell 60:941-951, 1990. Similarly, sequences of IL-15 from human and other species were also reported in the literature. For example, human, simian and mouse IL-15 cDNA, as well as human and mouse IL-15 genomic clones, have been isolated and characterized. See, e.g., Grabstein et al., Science 264:965-8, 1994; Anderson et al., Genomics 25:701-6, 1995; Bamford et al. Cytokine 7:595, 1995; and Giri et al., J. Leukoc. Biol. 57:763-766, 1995. The IL-15 cDNA clones from all three species encode a 162 amino acid (aa) residue precursor protein containing a 48 aa residue leader that is cleaved to generate the 114 aa residue mature IL-15. Human IL-15 shares approximately 97% and 73% sequence identity with simian and mouse IL-15, respectively. Both human and simian IL-15 are active on mouse cells.

In addition, human IL-15 and mouse IL-15 receptor sequences are also known in the art. See, e.g., Giri et al., EMBO J. 14:3654-63, 1995; and Anderson et al., J. Biol. Chem. 270:29862-9, 1995. IL-15 receptor consists of an IL-15Rα subunit, and shares common beta chain (CD122) and gamma chain (CD132) with the IL-2 receptor. This receptor has been cloned and characterized. See, e.g., Anderson et al., J. Biol. Chem. 270:29862-9, 1995 and Giri et al., EMBO J. 15:3654, 1995. It was known that both human and simian IL-15 can bind to a complex of the human beta and gamma common chain subunits in the absence of the mouse IL-15R alpha subunit, and that simian IL-15 is not capable of binding to and transducing IL-15 signals through the mouse beta and gamma common chain complex alone. Soluble human IL-2R beta appears to bind human IL-15 with sufficiently high affinity such that it is an excellent IL-15 antagonist.

As described above, antagonist compounds of IL-7 or IL-15 to be employed in the practice of the invention can antagonize the target via a variety of ways. They include any compounds or substances that inhibit or neutralize a signaling activity mediated by IL-7 or IL-15. They also encompass compounds that suppress or decrease expression or cellular level of IL-7, IL-15 or the receptor. Thus, as detailed below, IL-7 or IL-15 antagonists suitable for the invention can be inhibitory polynucleotides or oligonucleotides such as siRNAs, shRNAs, antisense molecules and DNAzymes that are specific for the target gene sequence. They can also be antibodies or antibody fragments that specifically recognize the target protein or polypeptide. Suitable IL-7 or IL-15 antagonists may further include fragments of the cytokine polypeptide or muteins that bind to its receptor. For example, blockade of IL-7R can be mediated via soluble IL-7Rα fragments as reported in Hartgring et al., Arthritis Rheum. 60:2595-605, 2009.

In some preferred embodiments, antagonist compounds to be employed in the practice of the present invention are antibodies that specifically bind to IL-7 receptor or IL-15 receptor, e.g., monoclonal antibodies recognizing IL-7Rα chain (CD127), IL-15Rα or IL-15Rβ (CD122). The antibodies can be monoclonal or polyclonal. Suitable monoclonal antibodies targeting the receptors of IL-7 and IL-15 can be human antibodies or non-human antibodies (e.g., mouse or rabbit antibodies). Preferably, the antibodies employed in the practice of the present invention are chimeric antibodies, humanized antibodies or fully human antibodies. Antibodies to be used in the invention also include antibody fragments or antigen-binding fragments which contain the antigen-binding portions of an intact antibody that retain capacity to bind the cognate antigen (e.g., IL-7Rα). Examples of such antibody fragments include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an intact antibody; (v) disulfide stabilized Fvs (dsFvs) which have an interchain disulfide bond engineered between structurally conserved framework regions; (vi) a single domain antibody (dAb) which consists of a V_{H} domain (see, e.g., Ward et al., Nature 341:544-546, 1989); and (vii) an isolated complementarity determining region (CDR). Antigen-binding molecules derived from these antibody fragments can also be used, e.g., polyethyleneglycolated Fab′ fragment.

The antibodies can be generated using methods well known in the art. An exemplary antibody for IL-7R is described in the Examples below. Additional anti-IL-7R antibodies are well known in the art. See, e.g., Kunisawa et al., Eur. J. Immunol. 32:2347-55, 2002; Demirkiran et al., J. Immunol. 178:6066-72, 2007; Dallas et al., J. Exp. Med. 201:1361-6, 2005; Vondenhoff et al., Development 136:29-34, 2009. Specific monoclonal antibodies targeting IL-7Rα can also be obtained from a number of commercial suppliers. Examples include eBioRDR5 mAb from eBioscience, Inc. (San Diego, Calif.); p449 mAb GenWay Biotech, Inc. (San Diego, Calif. 92121); mAbs C-20, H-215, E-17 and P14 from Santa Cruz Biotechnology, Inc. (Santa Cruz, Calif.); and mAb hIL-7R-M21 from BD Biosciences (San Jose, Calif.). Similarly, many antibodies specific for IL-15Rα are also known in the art, some of which can also be purchased from commercial sources. Examples of mAb anti-IL-15Rα that can be employed in the present invention include antibodies clones E-14, 34593.11, B-E29, 500-M15, YNR-HIL15 and Z-13L available from Santa Cruz Biotechnology, Inc. Additional antibodies specific for IL-7Rα or IL-15R are available from R and D Systems (Minneapolis, Minn.) and US Biological (Swampscott, Mass.).

In general, the production of non-human monoclonal antibodies, e.g., murine or rat, can be accomplished by, for example, immunizing the animal with a human IL-7 or IL-15 receptor polypeptide using standard techniques as described in the art (See Harlow & Lane, Antibodies, A Laboratory Manual, Cold Spring Harbor Laboratory Press, Cold Spring Harbor N.Y., 2000). Such an immunogen can be obtained from a natural source, by peptides synthesis or by recombinant expression. Humanized forms of IL-7R or IL-15R antibodies can be generated by linking the CDR regions of non-human antibodies to human constant regions by recombinant DNA techniques. See Queen et al., Proc. Natl. Acad. Sci. USA 86, 10029-10033, 1989; and WO 90/07861. Human antibodies can be obtained using phage-display methods. See, e.g., Dower et al., WO 91/17271; McCafferty et al., WO 92/01047. Human antibodies against IL-7R or IL-15R can also be produced from non-human transgenic mammals having transgenes encoding at least a segment of the human immunoglobulin locus and an inactivated endogenous immunoglobulin locus. See, e.g., Lonberg et al., WO93/12227 (1993); Kucherlapati, WO 91/10741 (1991).

In some other embodiments, expression or cellular level of IL-7 or IL-15 is suppressed or downregulated with inhibitory polynucleotides or nucleic acid molecules that specifically target an IL-7 or IL-15 mRNA transcript. Such inhibitory polynucleotide molecules can be complementary, DNAzymes, antisense molecules, double stranded homologues, short interfering RNA (siRNA) molecules, or sequence specific single-stranded RNAs which form short hairpin structures, shRNA. In some preferred embodiments, the employed inhibitory polynucleotides are siRNA molecules. siRNA molecules are short (preferably 19-25 nucleotides; most preferably 19 or 21 nucleotides), double-stranded RNA molecules that cause sequence-specific degradation of a target mRNA. This degradation is known as RNA interference (RNAi) (see, e.g., Bass et al., Nature 411:428-29, 2001). Originally identified in lower organisms, RNAi has been effectively applied to mammalian cells. For example, RNAi has been shown to prevent fulminant hepatitis in mice treated with siRNAs targeted to Fas mRNA (Song et al., Nature Med. 9:347-51, 2003). In addition, intrathecally delivered siRNA has been reported to block pain responses in two disease models (agonist-induced pain model and neuropathic pain model) in the rat (Dorn et al., Nucleic Acids Res. 32:e49, 2004).

siRNA molecules suitable for the present invention can be generated by annealing two complementary single-stranded RNA molecules together (one of which matches a portion of the target mRNA) or through the use of a single hairpin RNA molecule that folds back on itself to produce the requisite double-stranded portion. These can be performed according to methods well known in the art, e.g., Fire et al., U.S. Pat. No. 6,506,559; and Yu et al., Proc. Natl. Acad. Sci. USA 99:6047-52, 2002. The siRNA molecules can be chemically synthesized (Elbashir et al., Nature 411:494-98, 2001) or produced by in vitro transcription using single-stranded DNA templates (Yu et al., supra). Alternatively, the siRNA molecules can be produced biologically, either transiently (Yu et al., supra; and Sui et al., Proc. Natl. Acad. Sci. USA 99:5515-20, 2002) or stably (Paddison et al., Proc. Natl. Acad. Sci. USA 99:1443-48, 2002), using an expression vector(s) containing the sense and antisense siRNA sequences.

Specific inhibitory polynucleotides targeting IL-7 and IL-15 or their receptors are known and have been employed by the skilled artisans in the art. See, e.g., Wittnebel et al., Cancer Res. 67:5594, 2007; Ozawa et al., J. Immunol. 173: 5180-5188, 2004; Sawa et al., Immunity 30:447-457, 2009; Mirghomizadeh et al., Exp. Cell Res. 315:3064-3075, 2009; Ozawa et al., Int'l Cong. Series 1284:175-180, 2005. Any of the specific inhibitory polynucleotides described in the art can be employed and modified in the practice of the present invention. In addition, inhibitory polynucleotides targeting IL-7 and IL-15 or their receptors can also be obtained from a number of commercial sources. For example, agents for RNA interference of IL-7 or IL-15 (e.g., siRNA oligonucleotides or shRNA vectors) can be obtained from or readily synthesized with reagents from commercial suppliers, e.g., Santa Cruz Biotechnology, Inc. (Santa Cruz, Calif.), Origene (Rockville, Md.), GeneCopoeia (Rockville, Md.), and Invitrogen (San Diego, Calif.). Examples include mouse IL-7 siRNA sc-39630, mouse IL-7 shRNA Plasmid sc-39630-SH, human IL-15 shRNA lentiviral particle sc-39645-V, human IL-15 siRNA sc-39645 from Santa Cruz Biotechnology, Inc. A number of shRNA constructs for targeting IL-7 or IL-15 are also available from Origene and GeneCopoeia (Rockville, Md.). Other specific examples of siRNAs targeting IL-7 are also described in the art, e.g., Sureban et al., Oncogene 27:4544-56, 2008. Additional siRNA molecules targeting IL-7 can be similarly designed and synthesized according to criteria and methods described herein or well known in the art. Any of these IL-7- or IL-15-specific siRNAs can all be employed in the practice of the present invention.

In addition to targeting IL-7 or IL-15 cytokines, some methods of the invention may utilize inhibitory polynucleotides to specifically target the cytokine receptors. For example, specific siRNAs or shRNA constructs targeting IL-7R or IL-15R can be employed in the practice of the invention. Such inhibitory polynucleotides are also available from a number of commercial sources, e.g., Origene, Santa Cruz Biotechnology and Sigma-Aldrich. Specific examples include 29 mer shRNA constructs against IL7R in pGFP-V-RS vector from Origene, IL-7R siRNA sc-35664 and IL-7R shRNA Plasmid sc-35664-SH from Santa Cruz Biotechnology, and shRNA constructs HSH009677 from GeneCopoeia. Similarly, siRNAs or shRNAs specific for IL-15R are also readily available from commercial vendors, e.g., siRNA IL-15Rα siRNA sc-40051 from Santa Cruz Biotechnology and shRNA constructs against IL15Rα from OriGene (Rockville, Md.).

Other than RNA interference via siRNA or shRNA, some embodiments of the invention can employ other inhibitory polynucleotides to target IL-7, IL-15 or their receptors. For example, DNAzymes can be used in the practice of the invention. DNAzymes are catalytic DNA molecules that are capable of cleaving either RNA (Breaker and Joyce, Chem. Biol. 1:223-9, 1994; and Santoro and Joyce, Proc. Natl. Acad. Sci. U.S.A. 94:4262-6, 1997) or DNA (Carmi et al., Chem. Biol. 3:1039-46, 1996) molecules. They are highly selective for the RNA sequence and as such can be used to down-regulate specific genes through, e.g., targeting the messenger RNA. Some other embodiments of the invention can employ antisense nucleic acid molecules that target IL-7, IL-15 or their receptors. These are polynucleotide molecules that are complementary to a sense nucleic acid encoding a target polypeptide such as IL-7, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid to be employed in the invention can be readily constructed using chemical synthesis and enzymatic ligation reactions using procedures known in the art. Suitable inhibitory polynucleotides for antagonizing IL-7 or IL-15 signaling also encompass ribozymes. Ribozymes are catalytic RNA molecules with ribonuclease activity that are capable of cleaving a single-stranded nucleic acid, such as an mRNA, to which they have a complementary region. A ribozyme having specificity for a target nucleic acid molecule can be designed and produced in accordance with standard procedures well known in the art. See, e.g., Cech et al., U.S. Pat. No. 4,987,071; Cech et al., U.S. Pat. No. 5,116,742; Haselhoff and Gerlach, Nature 334:585-591, 1988; and Bartel and Szostak, Science 261:1411-1418, 1993.

IL-7 or IL-15 antagonists suitable for practicing the present invention further include agents of other chemical classes, e.g., small molecule organic compounds. By modulating their effect on IL-7 or IL-15 signaling, such antagonists can be readily identified from a library of candidate agents with screening assays routinely practiced in the art. Candidate agents include unringed and unbranched small organic molecules, as well as other organic compounds such as aromatic compounds, heterocyclic compounds, and benzodiazepines. Candidate agents to be screened for additional IL-7 or IL-15 antagonist compounds can also include polypeptides, beta-turn mimetics, polysaccharides, phospholipids, hormones, prostaglandins, steroids, purines, pyrimidines, oligomeric N-substituted glycines, oligocarbamates, saccharides, fatty acids, as well as derivatives, structural analogs or combinations thereof.

In some embodiments, combinatorial libraries of small molecule candidate agents can be employed to screen for small molecule IL-7 or IL-15 antagonists. A number of specific assays are available for such screening, e.g., as described in Schultz et al., Bioorg. Med. Chem. Lett. 8:2409-2414, 1988; Weller et al., Mol. Divers. 3:61-70, 1997; Fernandes et al., Curr. Opin. Chem. Biol. 2:597-603, 1998; and Sittampalam et al., Curr. Opin. Chem. Biol. 1:384-91, 1997. The candidate agents can be screened for an activity to down-regulate IL-7 or IL-15 own expression, or to inhibit a downstream signaling activity mediated by IL-7 or IL-15. Various biochemical and molecular biology techniques or assays well known in the art can be employed to practice the screening methods of the present invention. Such techniques are described in, e.g., Handbook of Drug Screening, Seethala et al. (eds.), Marcel Dekker (1^{st} ed., 2001); High Throughput Screening: Methods and Protocols (Methods in Molecular Biology, 190), Janzen (ed.), Humana Press (1^{st} ed., 2002); Current Protocols in Immunology, Coligan et al. (Ed.), John Wiley. & Sons Inc (2002); Sambrook et al., Molecular Cloning: A Laboratory Manual, Cold Spring Harbor Press (3^{rd} ed., 2001); and Brent et al., Current Protocols in Molecular Biology, John Wiley & Sons, Inc. (ringbou ed., 2003).

### IV. Inhibiting Proliferation and Activation of T Lymphocytes

The invention provides methods for inhibiting proliferation and/or activation of different subsets of T lymphocytes by antagonizing IL-7 or IL-15 signaling. As disclosed herein, there is a persistent downregulation of IL-7 receptor and IL-15 receptor in overactivated T cells (e.g., CD4⁻CD8⁻ double negative T cells). Without the receptors to consume the cytokines, excess of IL-7 can support proliferation of the autoreactive T cells and progression of systemic autoimmunity. In addition, it was found that blockade of IL-7R reduced T cell activation and autoimmune manifestations. Accordingly, the invention provides methods for inhibiting or suppressing proliferation of overactivated T cells in a subject by antagonizing IL-7 signaling or IL-15 signaling, e.g., via blockade of the cytokine receptors. These therapeutic methods can find applications in treating subjects with systemic autoimmunity or related diseases.

As demonstrated in the Examples herein, the present inventors additionally discovered that IL-7 exerts a novel, distinct, and crucial costimulatory function in antigen-induced CD4⁺ T cell. In addition, blockade of IL-7Rα was shown to curtail the expansion and promote apoptosis of autoantigen-specific CD4⁺ T cells. Accordingly, the invention provides methods for inhibiting activation of CD4⁺ T lymphocytes. With these methods, activation of this subset of T lymphocytes in a subject can be similarly inhibited or suppressed by blockade of IL-7 signaling. These methods can also be employed in the treatment of various inflammatory or autoimmune diseases.

The therapeutic methods of the invention are typically used to treat subjects with diseases or disorders which are mediated by or associated with hyperactive immune responses or aberrant T lymphocyte activation or proliferation. In particular, subjects suffering from or at risk of developing various inflammatory disorders are suitable for application of the methods in order to derive therapeutic or prophylactic benefits. For example, the disclosed methods for inhibiting T cell activation or proliferation can be useful for treating subjects afflicted with autoimmune diseases, such as lupus, multiple sclerosis, neuromyelitis optica (NMO), Sjogren's syndrome, Crohn's disease (intestine destruction) and others. The methods are also applicable to treating other inflammatory disorders or conditions associated with undesired immune responses, e.g., allergic reactions, type 1 diabetes and transplantation rejections.

### V. Treating Disorders Associated with Hyperactive Immune Responses

The invention provides methods for prophylactic or therapeutic treatment of a number of disorders associated with hyperactive immune responses, including autoimmune disorders and other inflammatory disorders. Subjects to be treated with the methods can be those who have already developed and are suffering from a condition mediated by a hyperactive immune system. They can also be ones who have a propensity or are at risk of developing a such a condition. By antagonizing IL-7 or IL-15 signaling, therapeutic effect of the methods can depend on down-regulating expression or cellular levels of the proinflammatory cytokine or other factors that contribute to hyperactive immune responses (e.g., COX-2). Thus, the therapeutic methods of the invention can optionally measure expression or cellular level of one or more proinflammatory factors (e.g., IL-1, TNFα or COX-2) in the subject during the course of the treatment. The methods can also include monitoring symptoms of an inflammatory disorder in the subject to be treated during the treatment process. By comparing symptoms of the subject prior to the treatment, during the course of treatment, and at the conclusion of the treatment, efficacy of the treatment can be readily determined.

As described above, disorders suitable for treatment with methods of the practice of the present invention broadly encompass autoimmune disorders and other abnormalities or conditions that are associated with or caused by hyperactive immune system or other abnormally regulated inflammatory response. They comprise a large, unrelated group of disorders which underlie a variety of human diseases, including disorders directly caused by hyperactive immune responses such as autoimmune diseases (e.g., lupus and multiple sclerosis), graft rejections, allergic reactions (e.g., asthma), inflammatory conditions in the epidermis (eczema), rheumatoid arthritis, and hay fever. Specific examples of diseases that may be suitable for the therapeutic methods of the invention include, for example, autoimmune diseases, e.g. rheumatoid arthritis, systemic lupus erythematosus, hashimoto's thyroidis, multiple sclerosis, myasthenia gravis, neuromyelitis optica (NMO), diabetes type I or II and the disorders associated therewith, vasculitis, pernicious anemia, Sjoegren syndrome, uveitis, psoriasis, Graves ophthalmopathy, alopecia greata and others, allergic diseases, e.g. allergic asthma, atopic dermatitis, allergic rhinitis/conjunctivitis, allergic contact dermatitis, inflammatory diseases optionally with underlying aberrant reactions, e.g. inflammatory bowel disease, Crohn's disease or ulcerative colitis, intrinsic asthma, inflammatory lung injury, inflammatory liver injury, inflammatory glomerular injury, atherosclerosis, osteoarthritis, irritant contact dermatitis and further eczematous dermatitises, seborrhoeic dermatitis, cutaneous manifestations of immunologically-mediated disorders, inflammatory eye disease, keratoconjunctivitis, myocarditis or hepatitis, ischemia/reperfusion injury, e.g. myocardial infarction, stroke, gut ischemia, renal failure or hemorrhage shock, traumatic shock, cancer, e.g. breast cancer, T cell lymphomas or T cell leukemias, infectious diseases, e.g. toxic shock (e.g. superantigen induced), septic shock, adult respiratory distress syndrome. Detailed descriptions of these disease and conditions are provided in the art, e.g., Kacmarek et al., Int Anesthesiol Clin. 37: 47-64, 1999; Powell et al., Hosp Med 61: 470-4, 2000; Neuhof et al., Resuscitation 14: 23-32, 1986; and Firth et al., Aust N Z J. Med. 21: 893-901, 1991.

In some preferred embodiments, therapeutic methods of the invention are directed to treating lupus. Subjects with lupus can be identified by observing symptoms and formal diagnosis. Common initial and chronic complaints include fever, malaise, joint pains, myalgias, fatigue, and temporary loss of cognitive abilities. Because they are so often present with other diseases, these signs and symptoms are not part of the diagnostic criteria for SLE. When occurring in conjunction with other signs and symptoms, however, they are considered suggestive. Dermatological manifestations such as malar rash are present in many lupus patients.

Diagnosis of lupus can be performed with well established procedures and criteria. For example, one can make a diagnosis on the basis of the American College of Rheumatology (ACR) classification criteria. For the purpose of identifying patients for clinical studies, a person has SLE if any 4 out of 11 symptoms are present simultaneously or serially on two separate occasions. These symptoms include, e.g., serositis, oral ulcers, arthritis, photosensitivity, hematologic disorder, and positive antinuclear antibody test, renal disorder, immunologic disorder (e.g., anti-ds DNA and antiphospholipid antibody), neurologic disorder (e.g., seizures or psychosis), Malar rash (rash on cheeks), and discoid rash (red, scaly patches on skin that cause scarring).

Laboratory tests are routinely used to aid diagnosis of lupus. Examples include serologic tests such as antinuclear antibody (ANA) testing and anti-extractable nuclear antigen (anti-ENA) testing. Several techniques are used to detect ANAs. Clinically the most widely used method is indirect immunofluorescence. The pattern of fluorescence suggests the type of antibody present in the patient's serum. ANA screening yields positive results in many connective tissue disorders and other autoimmune diseases, and may occur in normal individuals. Subtypes of antinuclear antibodies include anti-Smith and anti-double stranded DNA (dsDNA) antibodies (which are linked to SLE) and anti-histone antibodies (which are linked to drug-induced lupus). Anti-dsDNA antibodies are highly specific for SLE; they are present in 70% of cases, whereas they appear in only 0.5% of people without SLE. The anti-dsDNA antibody titers also tend to reflect disease activity, although not in all cases. Other ANA that may occur in SLE sufferers are anti-U1 RNP (which also appears in systemic sclerosis), SS-A (or anti-Ro) and SS-B (or anti-La; both of which are more common in Sjögren's syndrome). SS-A and SS-B confer a specific risk for heart conduction block in neonatal lupus. Other tests routinely performed in suspected SLE are complement system levels (low levels suggest consumption by the immune system), electrolytes and renal function (disturbed if the kidney is involved), liver enzymes, and complete blood count.

Once a subject with lupus is diagnosed, an agent that specifically targets IL-7 or IL-15 signaling as disclosed herein can be administered. Detailed procedures and formulations for the therapeutic regimen are detailed below. While the methods can be employed alone in the treatment of the subject, the therapeutic methods can also be utilized in conjunction with other known treatment regimens. For example, other immunosuppressive drugs can also be used in conjunction with the methods of the present invention. In more severe cases, medications that modulate the immune system (primarily corticosteroids and immunosuppressants) are used to control the disease and prevent recurrence of symptoms (known as flares). Depending on the dosage, people who require steroids may develop Cushing's syndrome, side-effects of which may include obesity, puffy round face, diabetes mellitus, large appetite, difficulty sleeping and osteoporosis. Those side-effects can subside if and when the large initial dosage is reduced, but long-term use of even low doses can cause elevated blood pressure and cataracts.

Due to the variety of symptoms and organ system involvement with SLE, its severity in an individual must be assessed in order to successfully treat SLE. Mild or remittant disease can sometimes be safely left untreated. If required, nonsteroidal anti-inflammatory drugs and antimalarials may be used. For example, disease-modifying antirheumatic drugs (DMARDs) are used preventively to reduce the incidence of flares, the process of the disease, and lower the need for steroid use; when flares occur, they are treated with corticosteroids. DMARDs commonly in use are antimalarials such as plaquenil and immunosuppressants (e.g. methotrexate and azathioprine). Hydroxychloroquine is an FDA-approved antimalarial used for constitutional, cutaneous, and articular manifestations. Hydroxychloroquine has relatively few side effects, and there is evidence that it improves survival among people who have SLE. Cyclophosphamide is used for severe glomerulonephritis or other organ-damaging complications.

In some other embodiments of the invention, subjects suffering from multiple sclerosis are the intended recipients of the therapeutic regimens of the invention. There are several subtypes, or patterns of progression, which have been described for MS. Subtypes use the past course of the disease in an attempt to predict the future course. They are important not only for prognosis but also for therapeutic decisions. In 1996, the United States National Multiple Sclerosis Society standardized four subtype definitions: relapsing remitting, secondary progressive, primary progressive, and progressive relapsing. The relapsing-remitting subtype is characterized by unpredictable relapses followed by periods of months to years of relative quiet remission with no new signs of disease activity. Deficits suffered during attacks may either resolve or leave sequelae, the latter being more common as a function of time. This describes the initial course of 85-90% of individuals with MS. When deficits always resolve between attacks, this is sometimes referred to as benign MS. The relapsing-remitting subtype usually begins with a clinically isolated syndrome (CIS). In CIS, a patient has an attack suggestive of demyelination, but does not fulfill the criteria for multiple sclerosis. However only 30 to 70% of persons experiencing CIS later develop MS.

Secondary progressive MS (sometimes called “galloping MS”) is present in about 65% of those with an initial relapsing-remitting MS, who then begin to have progressive neurologic decline between acute attacks without any definite periods of remission. Occasional relapses and minor remissions may appear. The median time between disease onset and conversion from relapsing-remitting to secondary progressive MS is 19 years.

The primary progressive subtype refers to approximately 10-15% of individuals who never have remission after their initial MS symptoms. It is characterized by progression of disability from onset, with no, or only occasional and minor, remissions and improvements. The age of onset for the primary progressive subtype is later than for the relapsing-remitting, but similar to mean age of progression between the relapsing-remitting and the secondary progressive. In both cases it is around 40 years of age. Progressive relapsing MS describes those individuals who, from onset, have a steady neurologic decline but also suffer clear superimposed attacks. This is the least common of all subtypes.

In addition, atypical variants of MS with non-standard behavior have been described. These include Devic's disease, Balo concentric sclerosis, Schilder's diffuse sclerosis and Marburg multiple sclerosis; and there is debate on whether they are MS variants or different diseases. Multiple sclerosis also behaves differently in children, taking them more time to reach the progressive stage. Nevertheless they still reach it at a lower mean age than adults.

Subjects with any of these MS subtypes can be treated with methods of the present invention. For example, the methods can be used to eliminate or ameliorate symptoms associated with MS. Subjects with MS can suffer almost any neurological symptom or sign, including changes in sensation (hypoesthesia and paraesthesia), muscle weakness, muscle spasms, or difficulty in moving; difficulties with coordination and balance (ataxia); problems in speech (dysarthria) or swallowing (dysphagia), visual problems (nystagmus, optic neuritis, or diplopia), fatigue, acute or chronic pain, and bladder and bowel difficulties. Cognitive impairment of varying degrees and emotional symptoms of depression or unstable mood are also common. Uhthoffs phenomenon, an exacerbation of extant symptoms due to an exposure to higher than usual ambient temperatures, and Lhermitte's sign, an electrical sensation that runs down the back when bending the neck, are particularly characteristic of MS. The therapeutic regimens disclosed herein can be employed for treating or alleviate any of these symptoms in a suffering subject.

Similar to treating lupus, the therapeutic methods of the invention can also be used to treat MS patients in conjunction with known MS treatments. For example, MS suffering subjects receiving regiments of the invention can be simultaneously treated with interferon beta-1a (trade names Avonex™, CinnoVex™, ReciGen™ and Rebif™) and interferon beta-1b (U.S. trade name Betaseron™, or Betaferon™ in Europe and Japan). Other suitable medications, e.g., glatiramer acetate (Copaxone™), mitoxantrone and natalizumab (marketed as Tysabri™), can also be used. These medications can be delivered to the subjects by frequent injections, varying from once-per-day for glatiramer acetate to once-per-week (but intra-muscular) for Avonex. Natalizumab and mitoxantrone can be administered by IV infusion at monthly intervals. Other treatments that can be used in combination with the methods of the invention include, dietary regimens, herbal medicine, including the use of medical cannabis, hyperbaric oxygenation and self-infection with hookworm.

The therapeutic methods of the invention typically entail administering to a subject in need of treatment a therapeutically effective amount of an antagonist compound specific for IL-7 or IL-15 (e.g., a siRNA or an antibody). The antagonist compound can be any agent which down-regulates expression or cellular level of IL-7, or inhibits or blocks the cellular activity or function of IL-7, e.g., an antagonist antibody, a small organic compound or a nucleic acid agent. Subjects suffering from autoimmune diseases or other inflammatory diseases can be treated with one of these antagonist compounds alone. The subjects can also be treated with the antagonist compound in conjunction with other known agents or regiments for treating or alleviating symptoms of autoimmune diseases or other inflammatory diseases.

In some embodiments, the subjects are administered with a nucleic acid antagonist that blocks or inhibits expression of a gene that encodes or is required for expression of IL-7. As described above, IL-7 or IL-15 expression can be downregulated by a number of classes of inhibitory polynucleotide molecules, e.g., siRNAs, shRNAs, antisense molecules and DNAzymes. Some preferred embodiments of the invention employ siRNA or shRNAs based antagonism of IL-7 or IL-15. siRNA and shRNA both work via the RNAi pathway and have been successfully used to suppress the expression of genes. Synthetic siRNAs designed to specifically target IL-7 or IL-15 can be easily delivered to cells in vitro or in vivo. shRNAs are the DNA equivalents of siRNA molecules and have the advantage of being incorporated into the cells' genome and then being replicated during every mitotic cycle. Some other embodiments of the invention can use DNAzymes designed to target IL-7 expression. DNAzymes are catalytic DNA molecules that cleave single-stranded RNA. They are highly selective for the target RNA sequence and as such can be used to down-regulate specific genes through targeting of the messenger RNA.

In still some other embodiments, the subjects can be treated with antisense RNAs specific for IL-7 or IL-15. Such agents can down-regulate IL-7 or IL-15 expression because they comprise the complement of an IL-7 (or IL-15) RNA sequence and are able to hybridize under stringent conditions to the IL-7 mRNA sequence. Such antisense molecules typically comprise a total of about 5 to about 100 or more, more preferably about 10 to about 60 nucleotides, and have a sequence that is preferably complementary to at least a portion of IL-7 (or IL-15) mRNA.

### VI. Pharmaceutical Compositions and Administration

Antagonist compounds targeting IL-7 or IL-15 signaling (e.g., antibodies and inhibitory polynucleotides) and the other therapeutic agents disclosed herein can be administered directly to subjects in need of treatment. However, these therapeutic compounds are preferably administered to the subjects in pharmaceutical compositions which comprise the cytokine antagonist and/or other active agents along with a pharmaceutically acceptable carrier, diluent or excipient in unit dosage form. Accordingly, the invention provides pharmaceutical compositions comprising one or more of the cytokine antagonist compounds disclosed herein. The invention also provides a use of these cytokine antagonists in the preparation of pharmaceutical compositions or medicaments for treating the above described diseases or medical disorders wherein undesired inflammatory responses (e.g., autoimmunity in lupus) and T cell proliferation or activation (e.g., CD4⁻CD8⁻ T cells) are present. Therapeutic kits which comprise at least one of the cytokine antagonist compounds described herein and an instruction sheet for using the compound to treat autoimmune diseases and inflammatory disorders (e.g., lupus or multiple sclerosis) are also provided in the invention.

Pharmaceutically acceptable carriers are agents which are not biologically or otherwise undesirable. These agents can be administered to a subject along with a cytokine (e.g., IL-7) antagonist compound without causing any undesirable biological effects or interacting in a deleterious manner with any of the components of the pharmaceutical composition. The compositions can additionally contain other therapeutic agents that are suitable for treating inflammatory disorders. Pharmaceutically carriers enhance or stabilize the composition or facilitate preparation of the composition. Pharmaceutically acceptable carriers include solvents, dispersion media, coatings, antibacterial and antifungal agents, isotonic and absorption delaying agents, and the like that are physiologically compatible. The pharmaceutically acceptable carrier employed should be suitable for various routes of administration described herein. Additional guidance for selecting appropriate pharmaceutically acceptable carriers is provided in the art, e.g., Remington: The Science and Practice of Pharmacy, Mack Publishing Co., 20^{th} ed., 2000.

Pharmaceutical compositions of the invention can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for molecules of the invention include ethylene-vinyl acetate copolymer particles, osmotic pumps, implantable infusion systems, and liposomes. Formulations for inhalation may contain excipients, for example, lactose, or may be aqueous solutions containing, e.g., polyoxyethylene-9-lauryl ether, glycocholate and deoxycholate, or may be oily solutions for administration in the form of nasal drops, or as a gel.

A pharmaceutical composition containing a cytokine antagonist compound described herein and/or other therapeutic agents can be administered by a variety of methods known in the art. The routes and/or modes of administration vary depending upon the desired results. Depending on the route of administration, the active therapeutic agent may be coated in a material to protect the compound from the action of acids and other natural conditions that may inactivate the agent. Conventional pharmaceutical practice may be employed to provide suitable formulations to administer such compositions to subjects. Any appropriate route of administration may be employed, for example, but not limited to, intravenous, parenteral, transcutaneous, subcutaneous, intramuscular, intracranial, intraorbital, intraventricular, intracapsular, intraspinal or oral administration. Depending on the specific conditions of the subject to be treated, either systemic or localized delivery of the therapeutic agents may be used in the treatment.

The cytokine antagonists for use in the methods of the invention should be administered to a subject in an amount that is sufficient to achieve the desired therapeutic effect (e.g., eliminating or ameliorating symptoms associated with autoimmune responses) in a subject in need thereof. Typically, a therapeutically effective dose or efficacious dose of the cytokine antagonist is employed in the pharmaceutical compositions of the invention. Actual dosage levels of the active ingredients in the pharmaceutical compositions of the present invention can be varied so as to obtain an amount of the active ingredient which is effective to achieve the desired therapeutic response for a particular subject, composition, and mode of administration, without being toxic to the subject. The selected dosage level depends upon a variety of pharmacokinetic factors including the activity of the particular compositions of the present invention employed, the route of administration, the time of administration, and the rate of excretion of the particular compound being employed. It also depends on the duration of the treatment, other drugs, compounds and/or materials used in combination with the particular compositions employed, the age, gender, weight, condition, general health and prior medical history of the subject being treated, and like factors. Methods for determining optimal dosages are described in the art, e.g., Remington: The Science and Practice of Pharmacy, Mack Publishing Co., 20^{th} ed., 2000. Typically, a pharmaceutically effective dosage would be between about 0.001 and 100 mg/kg body weight of the subject to be treated.

The cytokine antagonist compounds (e.g., an anti-IL-7a antibody or an IL-7 specific siRNA) and other therapeutic regimens described herein are usually administered to the subjects on multiple occasions. Intervals between single dosages can be daily, weekly, monthly or yearly. Intervals can also be irregular as indicated by measuring blood levels of the cytokine antagonist compounds and the other therapeutic agents used in the subject. In some methods, dosage is adjusted to achieve a plasma compound concentration of 1-1000 μg/ml, and in some methods 25-300 μg/ml or 10-100 μg/ml. Alternatively, the therapeutic agents can be administered as a sustained release formulation, in which case less frequent administration is required. Dosage and frequency vary depending on the half-life of the cytokine antagonist compound and the other drugs in the subject. The dosage and frequency of administration can vary depending on whether the treatment is prophylactic or therapeutic. In prophylactic applications, a relatively low dosage is administered at relatively infrequent intervals over a long period of time. Some subjects may continue to receive treatment for the rest of their lives. In therapeutic applications, a relatively high dosage at relatively short intervals is sometimes required until progression of the disease is reduced or terminated, and preferably until the subject shows partial or complete amelioration of symptoms of disease. Thereafter, the subject can be administered a prophylactic regime.

### EXAMPLES

The following examples are provided to further illustrate the invention but not to limit its scope. Other variants of the invention will be readily apparent to one of ordinary skill in the art and are encompassed by the appended claims.

### Example 1

### MRL-Fas^{lpr} T Cells Exhibit Phenotypic Markers of Homeostatic Proliferation

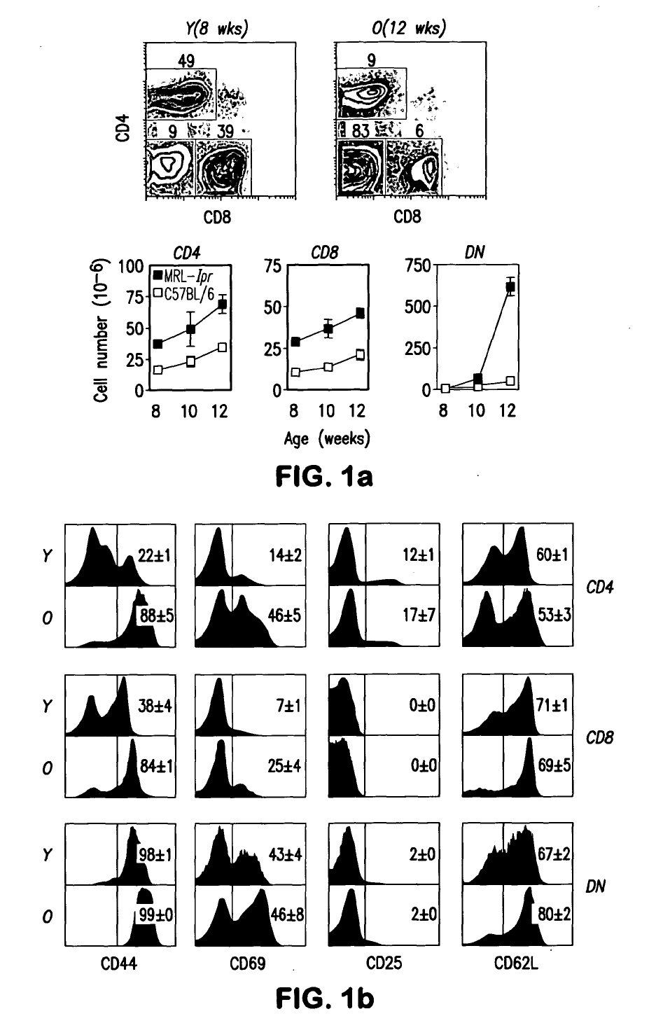

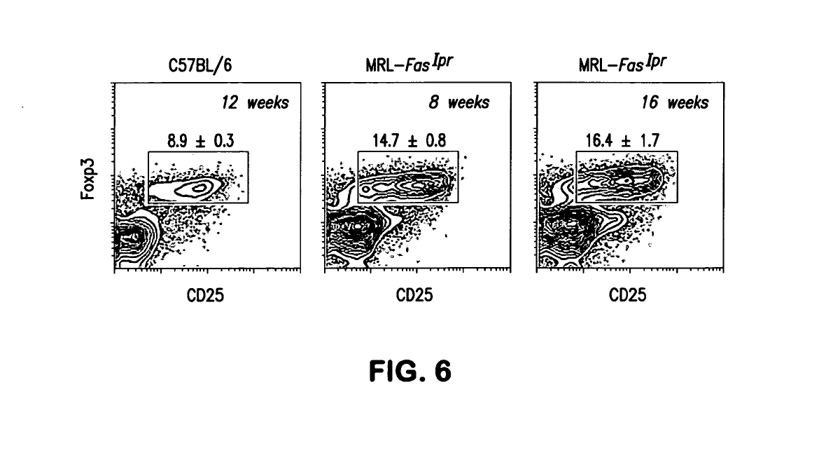

T cell activation in autoimmunity might be induced by either conventional antigen-mediated engagement or excess of T cell trophic cytokines and homeostatic proliferation. These two types of activation can be distinguished by differences in phenotypic markers, as cognate antigen-activated T cells are CD44^{hi}CD69⁺CD25⁺CD62L^{low}, whereas homeostatically-expanded T cells are CD44^{hi}CD69⁻CD25^{-l CD}62L^{hi 11,19}. We found that, with age and disease progression, MRL-Fas^{lpr} mice exhibited a moderate expansion of CD4⁺ and CD8⁺ single-positive (SP) T cells, and a much larger expansion of CD4⁻CD8⁻ double-negative (DN) T cells (FIG. 1a). Interestingly, substantial fractions of SP and DN T cells that accumulated in these mice were CD44^{hi}CD69⁻CD25⁻CD62L^{hi}, suggesting that expansion of these cells might be caused by excess of T cell-trophic cytokines (FIG. 1b). This interpretation is supported by the fact that the major activation marker CD25 was only expressed by the CD4⁺Foxp3⁺ regulatory T cell subset (FIG. 1b and FIG. 6). However, ˜50% of CD4⁺ and DN T cells, and ˜25% of CD8⁺ T cells were CD69^{hi}, indicating a concurrent conventional self or foreign antigen-driven activation.

### Example 2

### DN T Cells Lack Receptors for IL-7 and IL-15

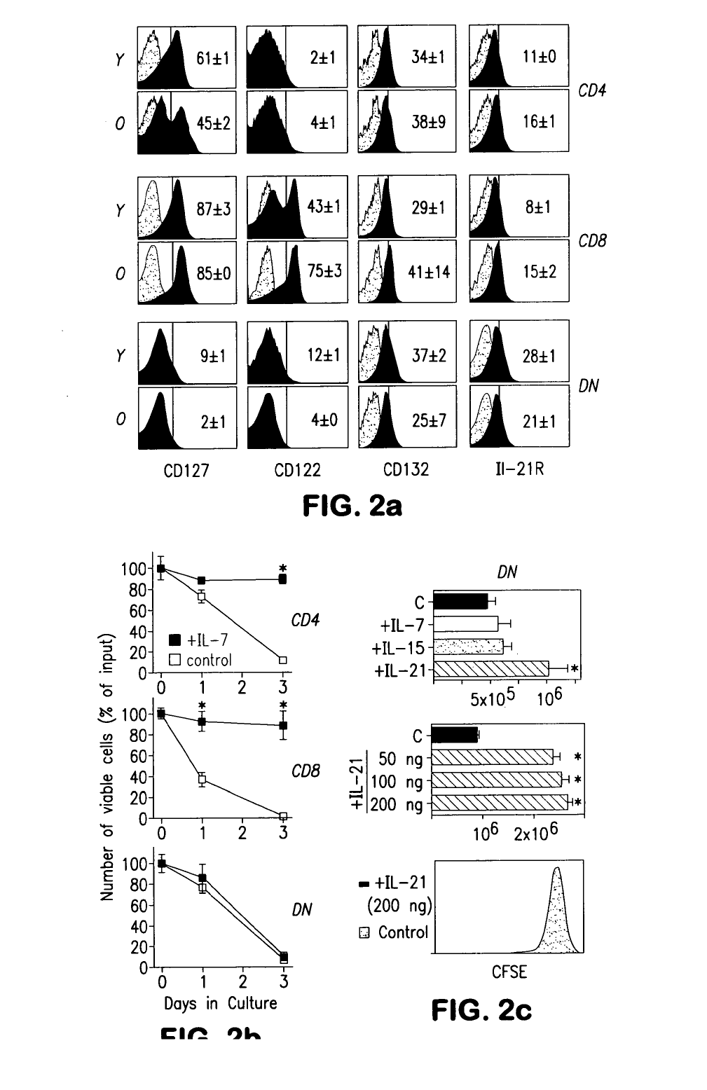

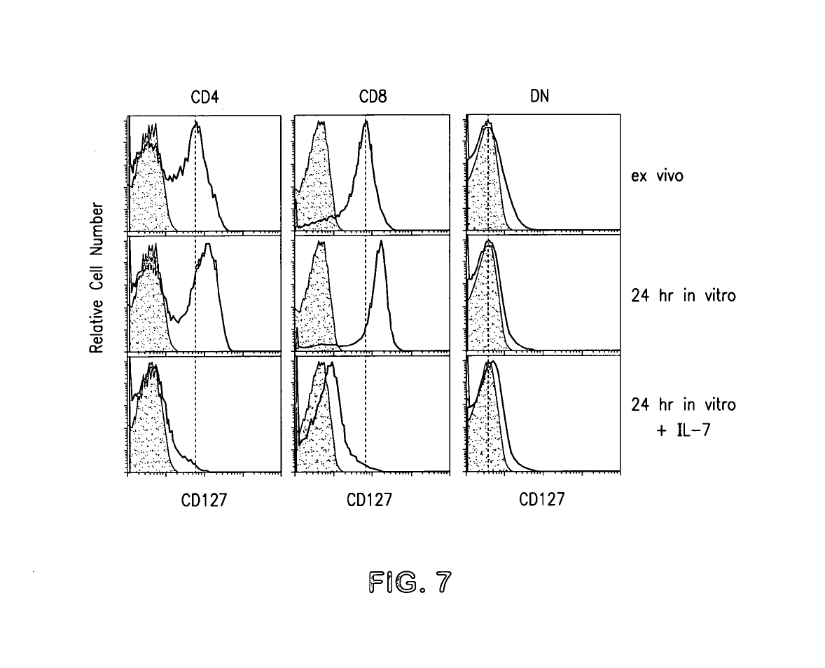

Because survival and homeostatic proliferation of T cells are dependent on signaling through IL-7R and IL-15R, we examined the expression profiles of these receptors. Depending on age, 45-61% of CD4⁺ T cells and 85-87% of CD8⁺ T cells in spleen and lymph nodes (LN) expressed high levels of CD127, while 43-75% of CD8⁺ T cells, but only 2-4% CD4⁺ T cells, expressed CD122 (FIG. 2a), frequencies similar to those in normal mice. Strikingly, however, virtually all DN T cells lacked both these receptors, particularly at advanced age (FIG. 2a). Accordingly, in vitro survival of CD4⁺ and CD8⁺ T cells was enhanced by IL-7, whereas DN T cells were not rescued by this cytokine (FIG. 2b). Moreover, CD127 was down-regulated in CD4⁺ and CD8⁺ T cells cultured in the presence of IL-7 and up-regulated in the absence of this cytokine, but receptor expression remained undetectable in DN T cells in either condition (FIG. 7), suggesting that CD127 is irreversibly down-regulated in DN T cells. Since most DN T cells are thought to derive from CD8⁺ precursors, we also examined the effects of IL-15 and IL-21, two cytokines known to support survival and proliferation of naïve and memory CD8⁺ T cells. Consistent with CD122 expression, IL-15 enhanced the in vitro survival of CD8⁺, but not DN, T cells. In contrast, IL-21 significantly enhanced survival of DN T cells without inducing proliferation (FIG. 2c), an effect that correlated with expression of both IL-21R chains (α and γc) by these cells (FIG. 2a). The results indicate that DN T cells lose CD127 and CD122, cease using IL-7 and IL-15, and switch to alternative survival resources, such as IL-21.

### Example 3

### Commensal Antigens Induce Conversion of CD8⁺ Precursors to DN T Cells

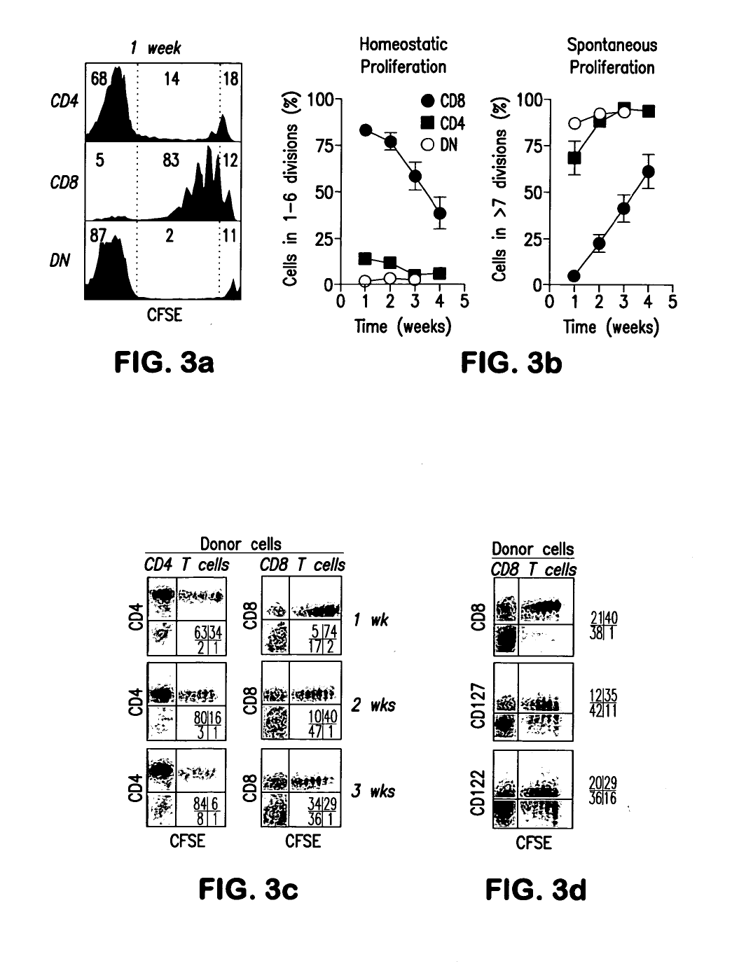

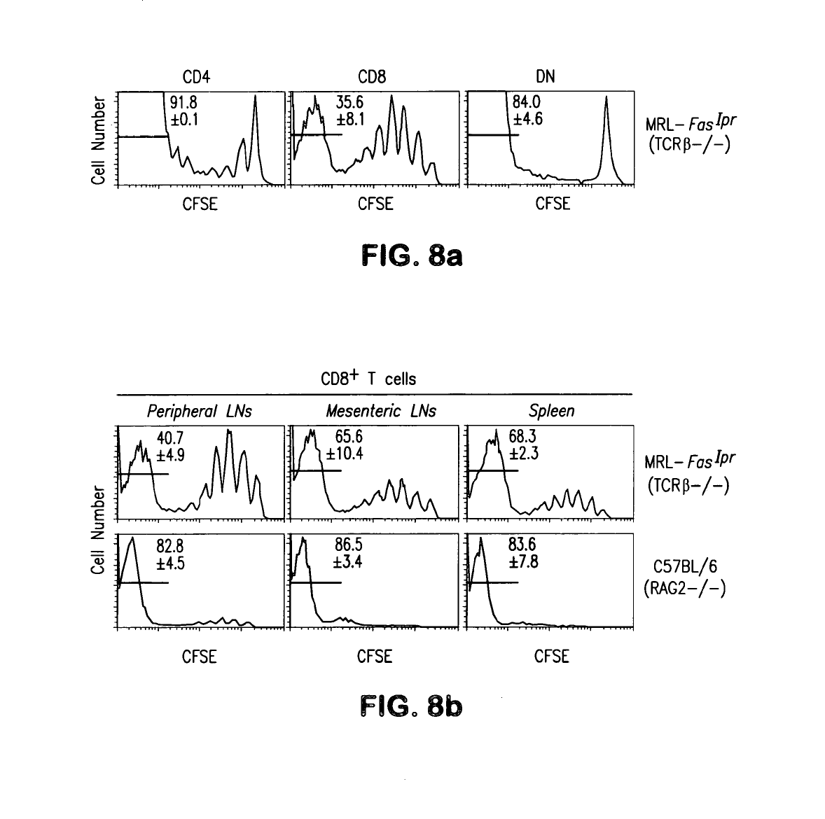

Persistent down-regulation of CD127 and CD122 has been observed in T cells undergoing extensive activation and reaching a functionally defined “exhausted” state. Although accumulation of DN T cells in MRL-Fas^{lpr} mice is due to defective Fas-mediated apoptosis, the nature of the antigens (self or foreign) that drive activation of the CD8⁺ precursors for DN cells has not been defined. To differentiate between self and foreign stimuli, experiments were performed with purified T cell subsets transferred into TCRβ^{-/-} MRL-Fas^{lpr} recipients. In these chronically immunodeficient hosts, T cells with slow division, defined as “homeostatic proliferation”, are thought to respond to self-peptide/MHC ligands, whereas those with fast division, defined as “spontaneous proliferation”, are thought to proliferate in an IL-7-independent manner, likely in response to intestinal flora- and/or food-derived antigens. As observed in chronically T cell-deficient normal background mice, at 7 days post-transfer of syngeneic T cells into TCRβ^{-/-} MRL-Fas^{lpr} recipients, ˜14% of CD4⁺ T cells in peripheral LNs (a pool of inguinal, brachial and axillary) had undergone slow-paced homeostatic proliferation (1 to 7 divisions) and ˜68% fast-paced spontaneous proliferation (>7 divisions) (FIG. 3a). Remarkably, however, the vast majority of dividing CD8⁺ T cells exhibited homeostatic proliferation, whereas DN T cells showed the opposite, i.e. almost exclusively spontaneous proliferation (FIG. 3a). These patterns were maintained even at 2 to 4 weeks post-transfer (FIG. 3b and FIG. 8a). Interestingly, spontaneous proliferation of CD8⁺ T cells was higher (˜1.6- to 2.2-fold at 2 weeks post-transfer) in mesenteric LNs, the primary site of commensal antigen recognition, and in spleen, but remained significantly lower than spontaneous proliferation of normal background CD8⁺ T cells transferred into chronically immunodeficient syngeneic recipients (FIG. 8b).

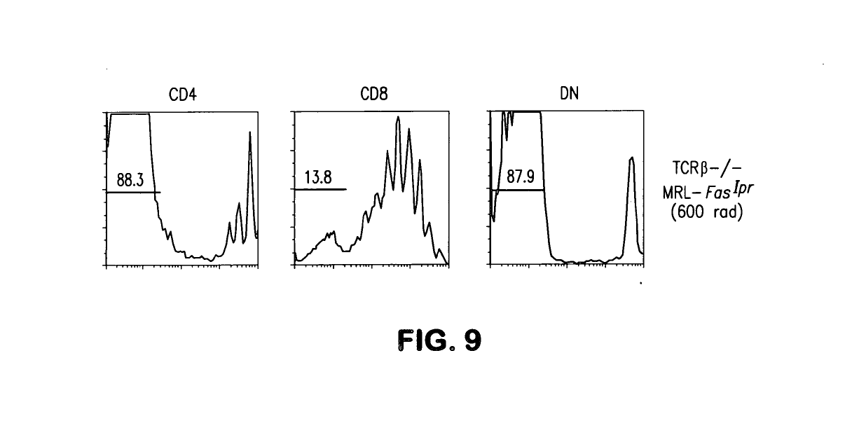

The reduction of spontaneously proliferating CD8⁺ T cells transferred into chronically immunodeficient MRL-Fas^{lpr} recipients was not due to lack of CD4⁺ T cells or competition with other cell types such as NK and γδ T cells, since slow division of CD8⁺ T cells was maintained when total LN cells were transferred into sublethally-irradiated TCRβ^{-/-} recipients (FIG. 9). Instead, reduced spontaneous proliferation could be due to conversion of CD8⁺ T cells to DN cells. Indeed, a significant proportion (51-82%) of CD8⁺ T cells that underwent >7 divisions in TCRβ^{-/-} recipients converted to DN T cells (FIG. 3c) and down-regulated both CD127 and CD122 (FIG. 3d). In contrast, only 3-9% of CD4⁺ T cells that had entered >7 divisions converted to DN T cells. Moreover, this conversion was not observed in T cells undergoing slow-paced (homeostatic) proliferation (FIGS. 3c and 3d). Hence, recognition of commensal antigens leads to strong proliferation of CD8⁺ T cells, together with down-regulation of the CD8 coreceptor and receptors for the major T cell trophic cytokines.

### Example 4

### Accumulation of DN T Cells Leads to Excess IL-7

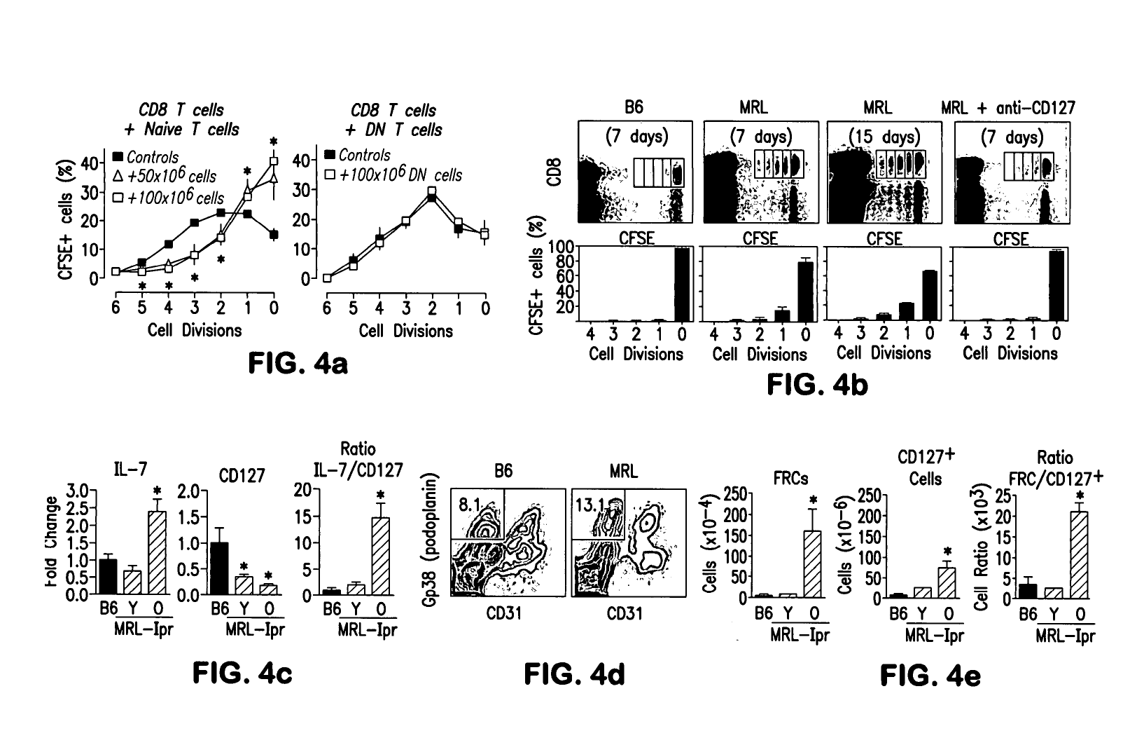

Down-regulation of CD 127 should make DN T cells inefficient consumers of trophic cytokines and therefore unable to inhibit T cell homeostatic proliferation of SP T cells in lymphopenic hosts. Moreover, it may create a sufficient excess of IL-7 that could provoke proliferation of T cells under non-lymphopenic conditions. Indeed, homeostatic proliferation of CFSE-stained MRL-Fas^{lpr} CD4⁺ and CD8⁺ T cells in TCRβ^{-/-} recipients was not inhibited by co-transfer of large numbers (100×10⁶) of purified DN T cells, in contrast to efficient inhibition by total LN T cells (50×10⁶) from young MRL-Fas^{lpr} donors in which there is only a minor DN T cell expansion (FIG. 4a). More importantly, a significant fraction of SP T cells proliferated when transferred into unmanipulated (non-lymphopenic) older MRL-Fas^{lpr} mice that had significant lymphadenopathy due to DN T cell expansion, and this proliferation was inhibited with an anti-IL-7Rα antibody (FIG. 4b). Contrastingly, no proliferation was detected in lymphosufficient normal recipients of syngeneic T cells. These findings indicate that, with age, MRL-Fas^{lpr} mice develop excess of IL-7 of sufficient magnitude to provoke proliferation of potentially autoreactive T cells.

### Example 5

### Expansion of IL-7-Producing Stromal Cells

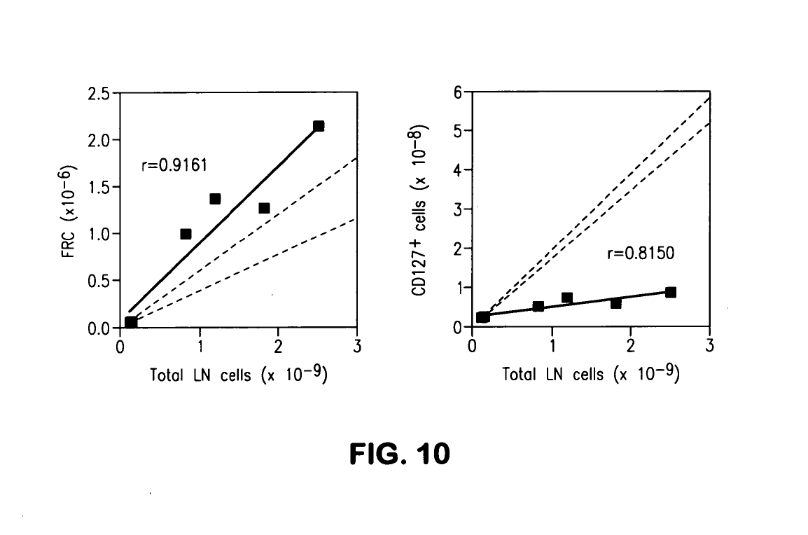

Because lymphadenopathy may also lead to the expansion of cells that produce IL-7, we examined expression levels and frequency of fibroblastic reticular cells (FRCs), the major producers of this cytokine in secondary lymphoid organs. There was a significant increase in IL-7 transcripts in the enlarged LNs of older MRL-Fas^{lpr} mice (FIG. 4c), while CD127 transcripts were decreased, thereby resulting in an 7-fold increase in the ratio of IL-7 to CD127 expression in older compared to younger mice (FIG. 4c). This result correlated with an 7.6-fold increase in the ratio between the numbers of FRCs and CD127⁺ T cells (FIG. 4d-e). Analysis of MRL-Fas^{lpr} mice with various degrees of lymphadenopathy indicated that FRCs accumulated proportionally to the number of total LN cells, whereas CD127⁺ T cells accumulated at much slower pace (FIG. 10). Thus, excess of IL-7 in MRL-Fas^{lpr} mice is caused by a combination of decreased consumption due to receptor down-regulation by DN T cells, and increased production due to FRC expansion.

### Example 6

### Anti-CD127 Antibody Treatment Reduces Disease in MRL-Fas^{lpr} Mice

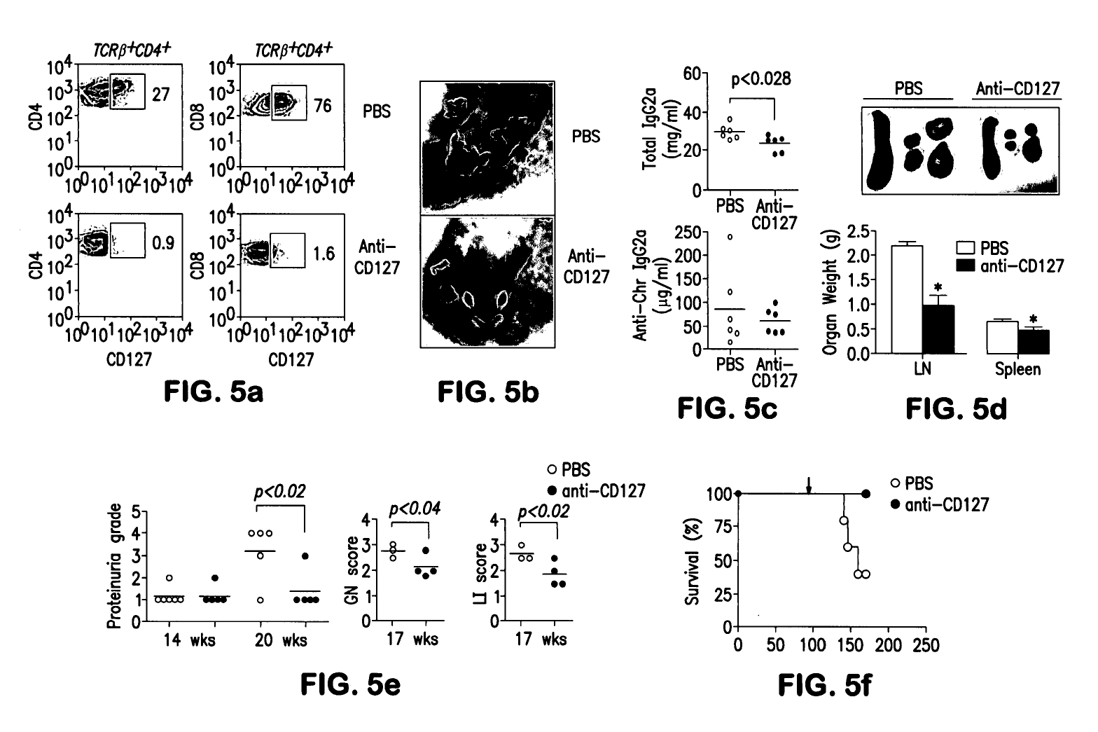

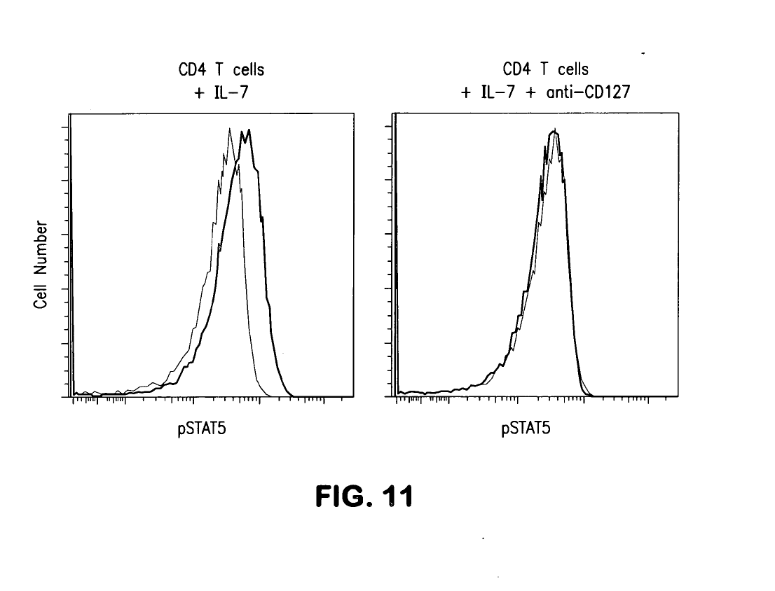

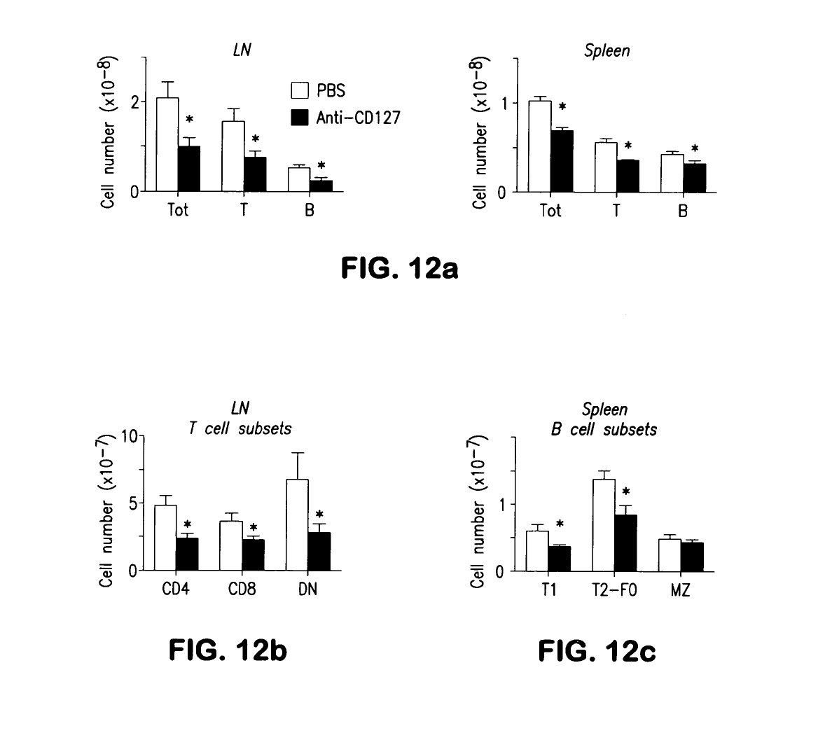

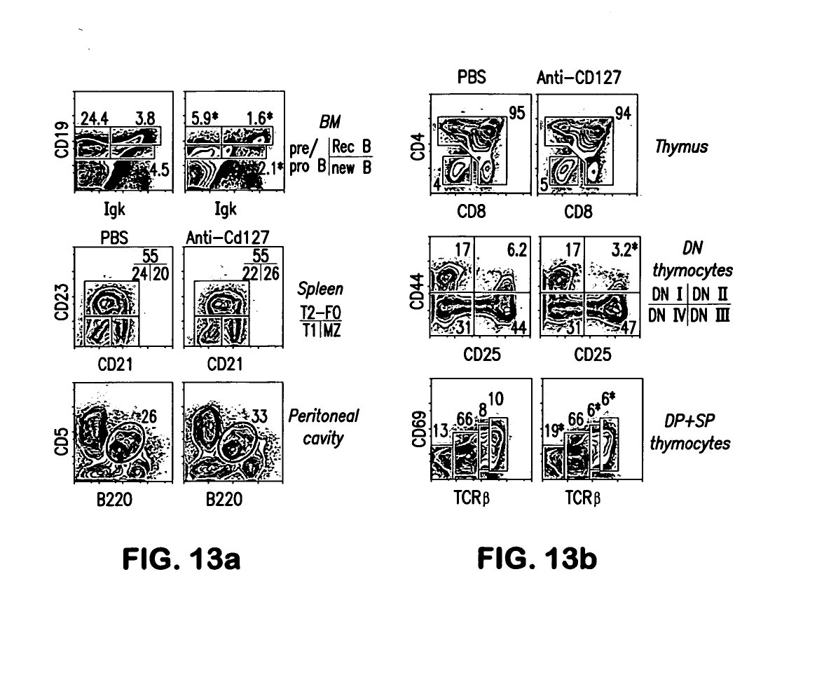

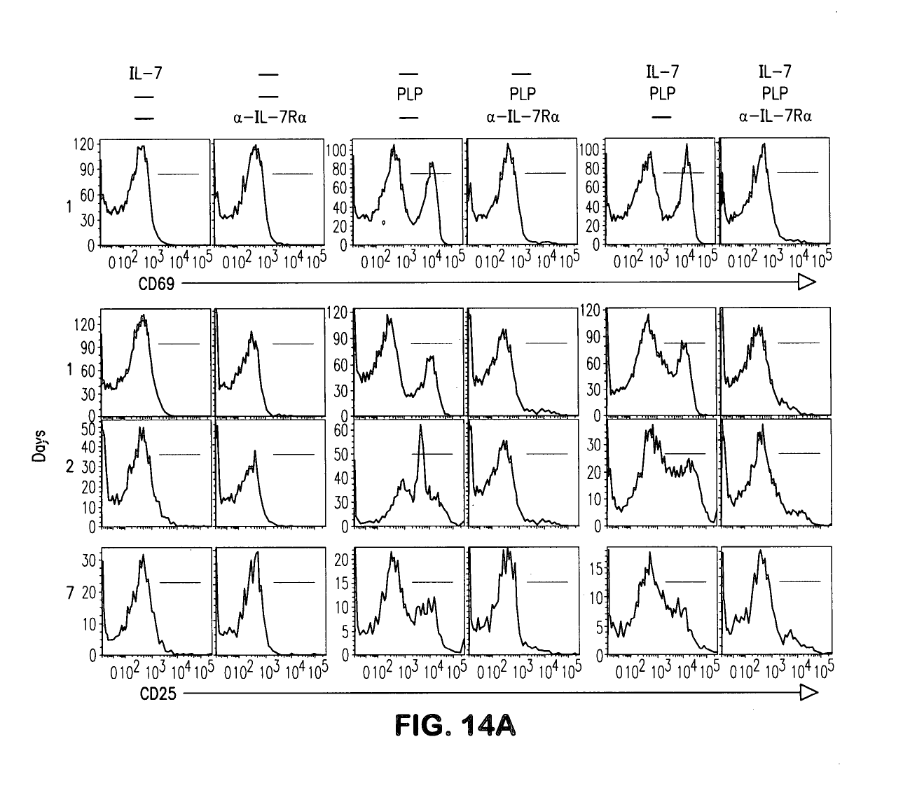

Because excess IL-7 signaling may decrease the activation threshold and provoke proliferation of autoreactive T cells, we examined whether blockade of IL-7R signaling could exert therapeutic effects in the MRL-Fas^{lpr} disease. Initial experiments showed that an anti-CD 127 monoclonal antibody (A7R34, rat IgG2a) effectively blocked this receptor in vivo (FIG. 5a), and inhibited IL-7-mediated STAT5 phosphorylation in vitro (FIG. 11). In vivo, this antibody was effective for up to 4 weeks, with progressive declines thereafter due to immune response against the heterologous antibody. Nonetheless, treatment initiated at early disease stages (6 weeks of age) and terminated 8 to 10 weeks later resulted in significant reduction of dermatitis, lymphadenopathy, splenomegaly and total serum IgG2a, as well as marginal reduction of anti-chromatin IgG2a autoantibodies, compared to PBS-injected controls (FIG. 5b-d). Treated mice also displayed reduced numbers of CD4⁺, CD8⁺and DN T cells, as well as immature (T1) and follicular (T2-F0) B cells, whereas marginal zone B cells and peritoneal B-1 cells were unaffected (FIG. 12). Moreover, there were reductions at the early developmental stages of B cells in the bone marrow and T cells in the thymus (FIG. 13).

A separate group of mice was then similarly treated beginning at 14 weeks of age to assess whether IL-7R blockade could inhibit established disease. Indeed, treatment significantly reduced proteinuria, glomerulonephritis, and lymphocyte infiltrates in the kidneys (FIG. 5e). Impressively, all antibody-treated mice were alive, whereas >50% of the controls had died, by 24 weeks of age when the experiment was terminated (FIG. 5f).

### Example 7

### IL-7 in CD4⁺ T Cell Activation and Neuroinflammation

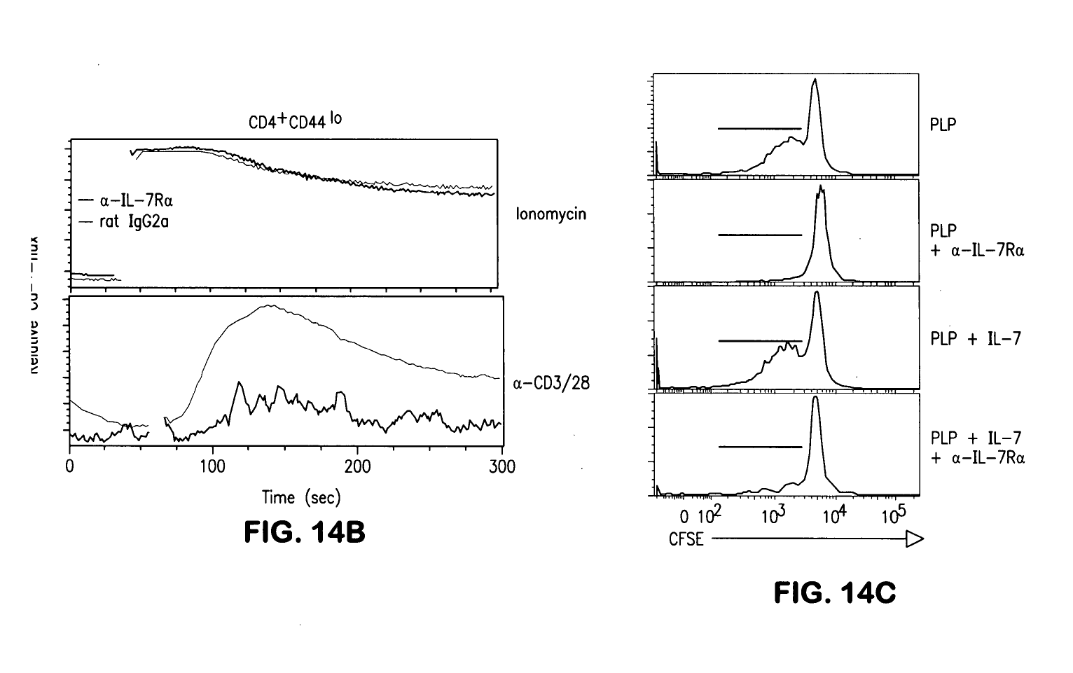

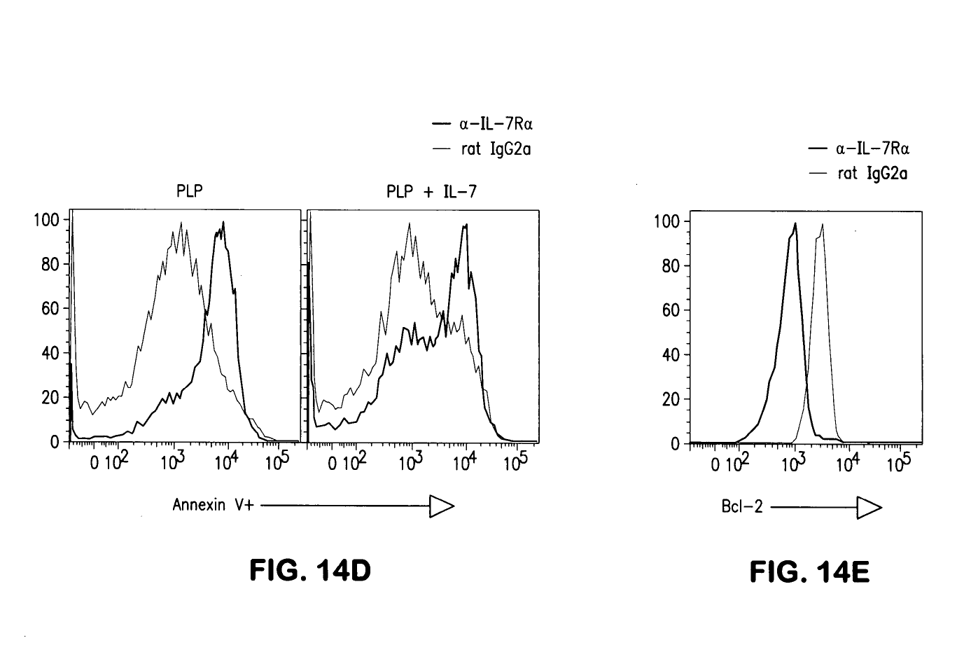

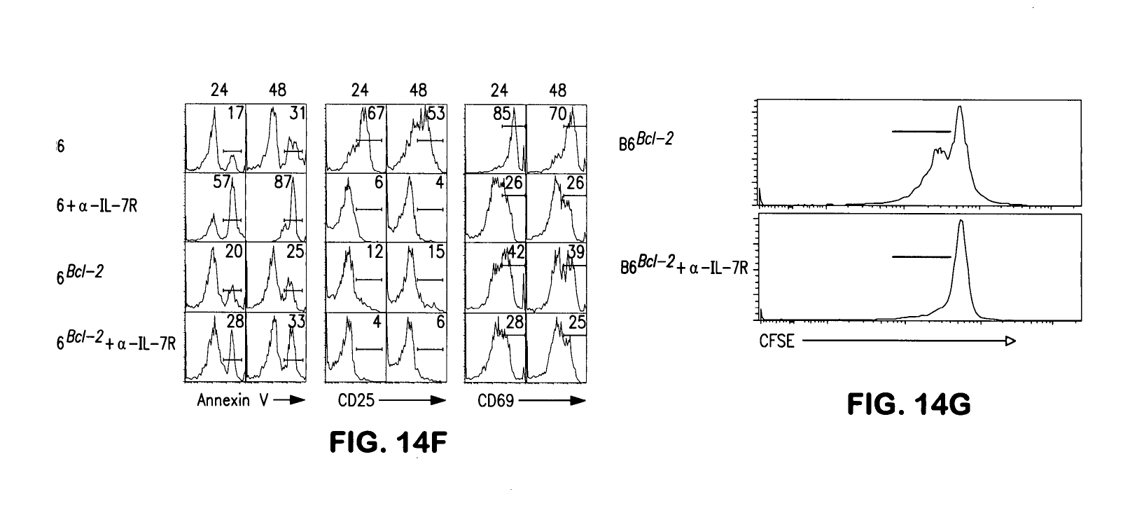

To evaluate the role of IL-7 during the initial stages of antigen-induced T cell activation, total splenocytes from proteolipid peptide (PLP)-specific TCR transgenic mice were treated for 1 hr with either a blocking anti-IL-7Rα (A7R34) or an isotype control antibody (anti-KLH) then stimulated with PLP in the presence or absence of IL-7. Unpredictably, treatment with an anti-IL-7Rα antibody significantly inhibited PLP-induced upregulation of activation markers (CD69 and CD25) (FIG. 14A), Ca²⁺ mobilization (FIG. 14B), and proliferation (FIG. 14C) of CD4⁺ T cells compared to controls. This treatment also promoted apoptosis (FIG. 14D) and inhibited upregulation of Bcl-2 (FIG. 14E). Addition of the anti-IL-7Rα antibody >24 hrs post-TCR stimulation, however, did not hinder expression of CD69 and CD25 (not shown). Similar results were obtained using OVA-specific OT-II TCR transgenic CD4⁺ T cells (not shown). These initial findings suggested that IL-7 is required at the earliest stages of TCR engagement for efficient CD4⁺ T cell activation.

To test whether IL-7RA blockade inhibited T cell activation by simply interfering with the prosurvival effects of IL-7, we performed the above experiments with total splenocytes from Bcl-2 transgenic mice. Surprisingly, although anti-CD3/CD28 activated Bcl-2 transgenic CD4⁺ T cells were protected from IL-7R blockade-induced apoptosis, they still failed to upregulate activation markers and to proliferate (FIGS. 14F and 14G). This further supports the conclusion that IL-7, in addition to promoting survival, also provides a vital costimulatory signal.

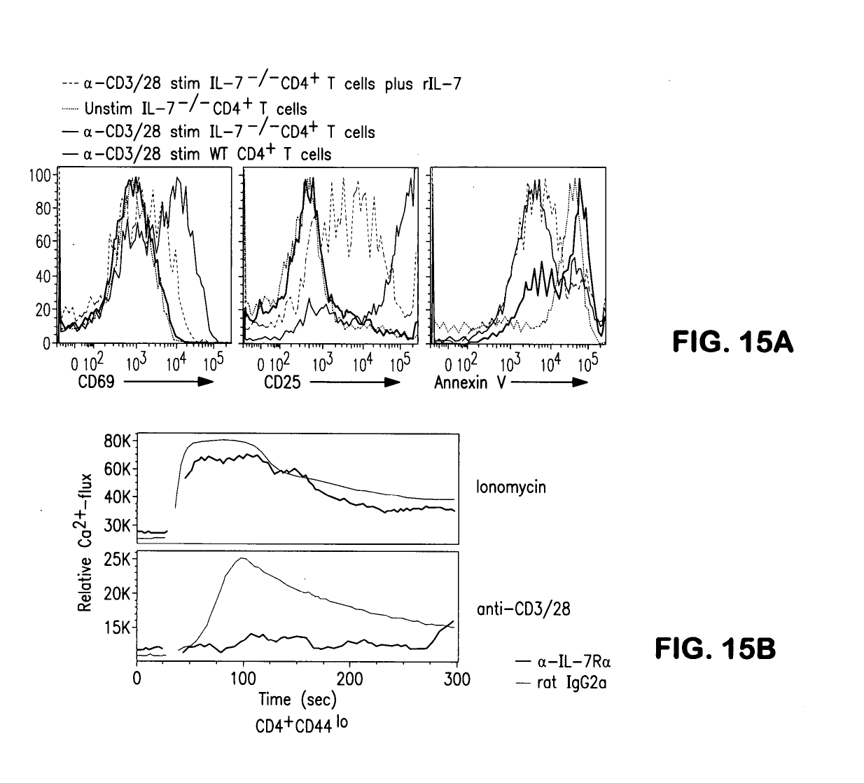

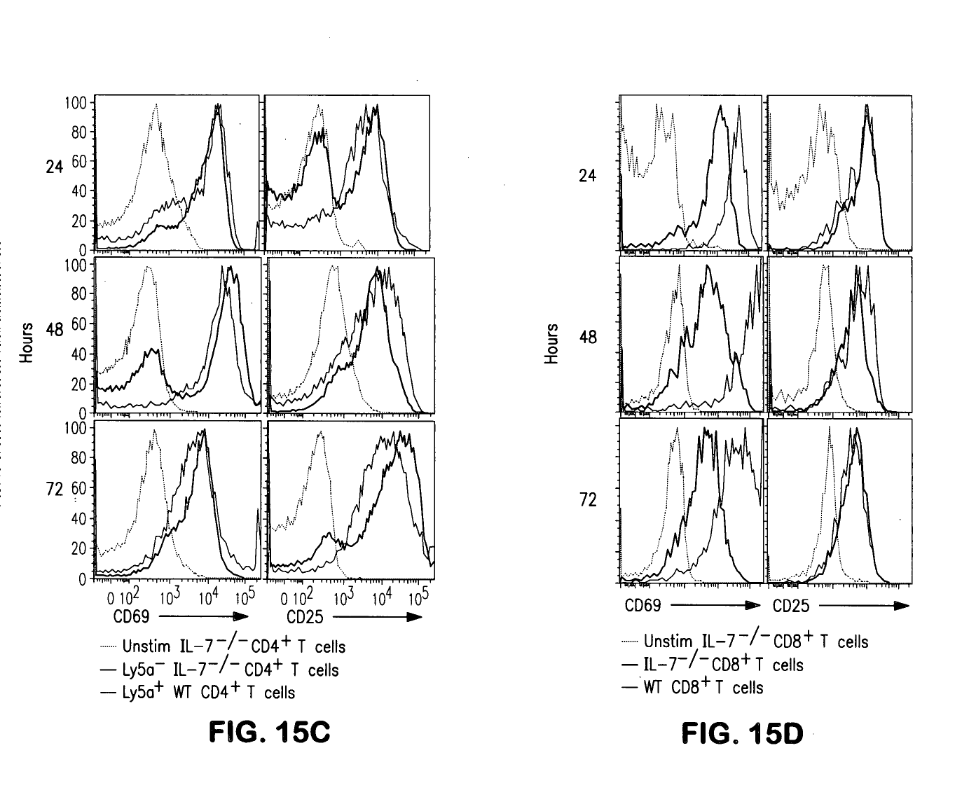

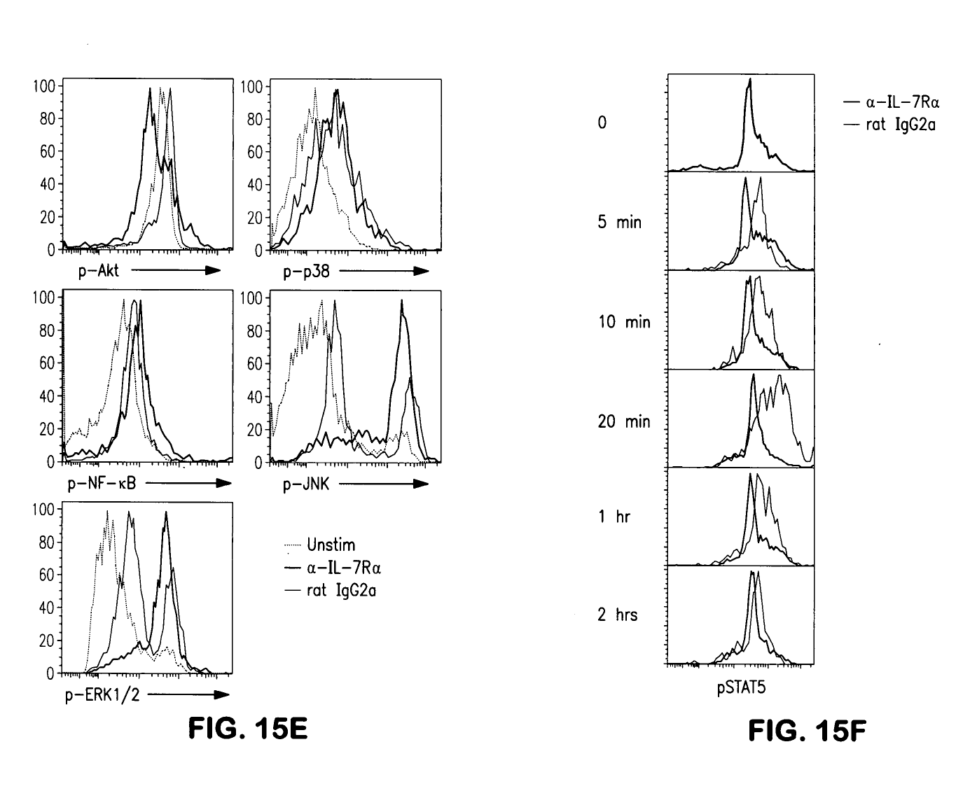

Next, we analyzed activation responses to anti-CD3/CD28 antibodies by non-transgenic CD4⁺ T cells from IL-7-deficient mice. At 48 hrs post-stimulation, there was strong upregulation of CD69 and CD25 by wild-type, but not IL-7^{-/-}, CD4⁺ T cells (FIG. 15A, left and middle panels). Similarly, TCR-stimulated IL-7 CD4⁺ T cells did not mobilize Ca²⁺ (FIG. 15B), and most of these cells were preapoptotic (Annexin V⁺) 48 hrs post-stimulation (FIG. 15A, right panel). The addition of recombinant IL-7 partially restored expression of the activation markers by these cells and prevented apoptosis (FIG. 15A). Furthermore, when mixtures of total splenocytes from IL-7^{-/-} (Ly5a⁻) and wild-type (Ly5a⁺) mice were stimulated with anti-CD3/CD28 antibodies, there was an equal and significant upregulation of the activation markers in both CD4⁺ T cell populations (FIG. 15C). Thus, the unresponsiveness of CD4⁺ T cells from IL-7 mice was not conferred by maturation of these cells in an IL-7-deficient environment, but lack of IL-7-mediated costimulation which was provided by the wild-type splenocytes. In contrast, CD8⁺ T cells from IL-7 mice upregulated CD25 normally following TCR-activation (FIG. 15D) and proliferated upon mitogen-induced stimulation (not shown). Yet, upregulation of CD69 was incomplete in these cells, supporting their primary dependency on IL-15 and not IL-7. Overall, the cumulative results, strongly suggested that IL-7 signaling is necessary for TCR-induced CD4⁺, but not CD8⁺, T cell activation.

Reliance of CD4⁺ T cell activation on IL-7 may be mediated by certain overlapping components within the signaling cascades initiated by the TCR and the IL-7R. Among these, likely candidates are members of the PI3K/Akt and Jak/STAT pathways. Essentially, activation of PI3K triggers both Ca²⁺ mobilization via activation of Tec family kinases and PLCγ, and, through Akt, promotes survival by inducing anti-apoptotic factors such as Bcl-2. On the other hand, activation of Jaks1 and 3 leads to phosphorylation and translocation of STAT5 to the nucleus which upregulates several genes critical for T cell activation, such as Cd69 and Il2ra. Strikingly, with anti-IL-7Rα antibody pretreatment, PLP-stimulated TCR transgenic CD4⁺ T cells showed persistent (up to 2 hrs) p38, JNK, and Erk1/2 hyperphosphorylation, yet Akt activation was markedly reduced (FIG. 15E), a phenotype suggestive of cells highly stressed due to IL-7 withdrawal. In contrast, cells pretreated with the isotype control antibody showed transient phosphorylation of Akt, p38, JNK, and Erk1/2 which, as expected, returned to basal levels by 40-60 min. There was no difference in NF-κBp65 phosphorylation, however, in both treatment groups, indicating that this TCR-signaling pathway was engaged and still functional. In addition, anti-IL-7Rα antibody strongly inhibited TCR-induced STAT5-phosphorylation (FIG. 15F). Thus, IL-7RA blockade impairs T cell activation and proliferation by inhibiting the PI3K/Akt cascade as well as STAT5 phosphorylation. In regards to the latter, it was previously reported that TCR engagement induced STAT5 phosphorylation by a CD3ζ-Lck-dependent process and, furthermore, that activated STAT5 was required for antigen-induced T cell proliferation. The present results, however, indicate that phosphorylation of STAT5 is provided by an IL-7R signal concurrent with TCR engagement. Thus, distinct IL-7 signaling pathways mediate CD4⁺ T cell activation as well as protection from activation-induced cell death (AICD).

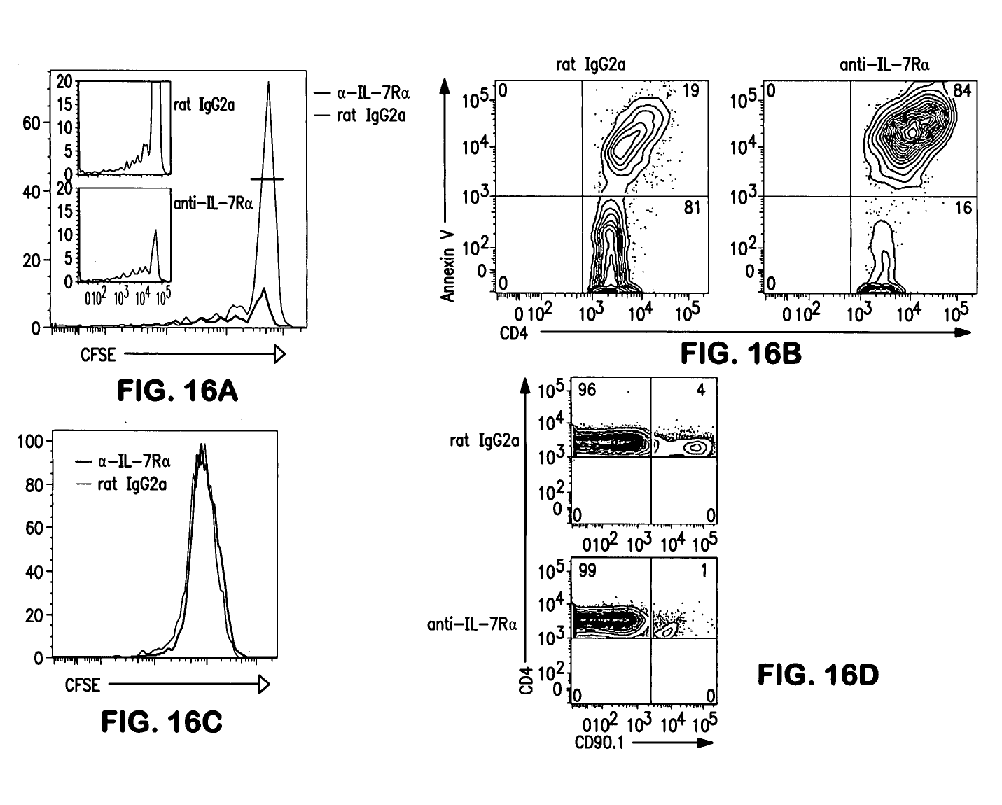

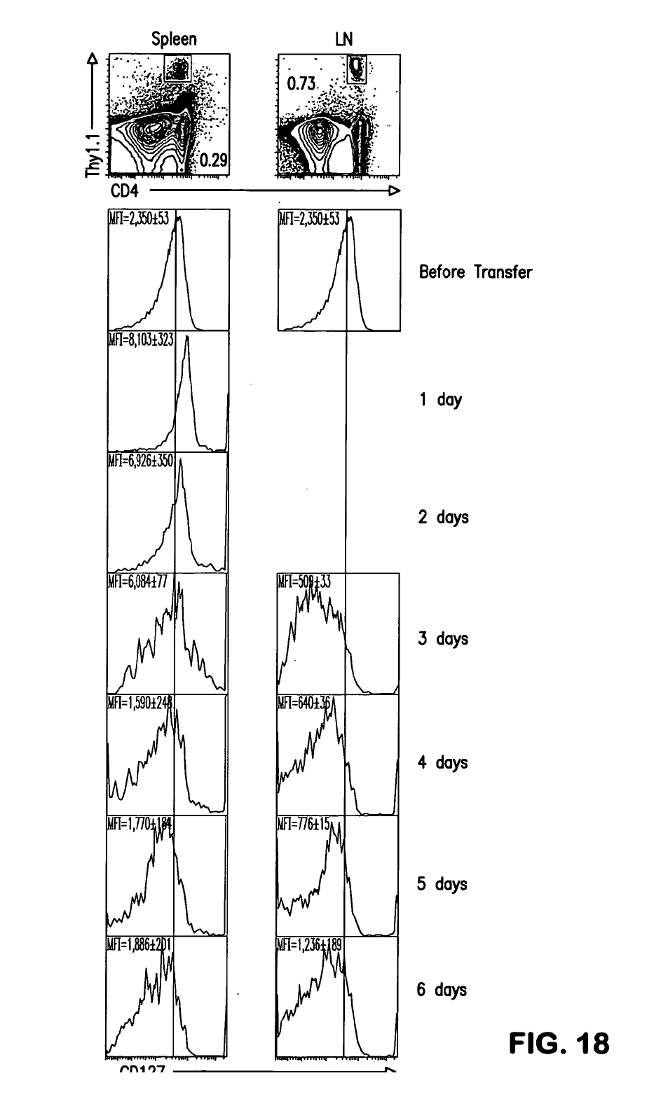

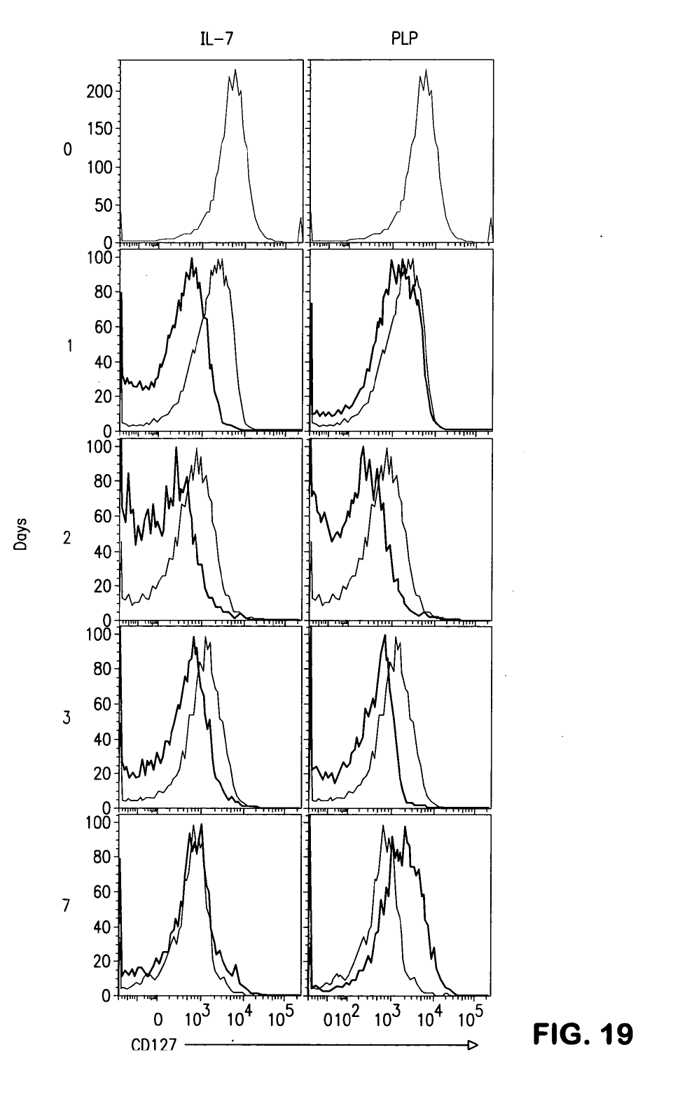

The in vitro data indicated that IL-7RA blockade reduced CD4⁺ T cell activation and promoted apoptosis, thereby suggesting that blockade of this receptor would likely interfere with ongoing in vivo immune responses, including those mediated by self-antigens. To examine these potential in vivo effects, we utilized the experimental autoimmune encephalomyelitis (EAE) mouse model of multiple sclerosis (MS) in which self-reactive CD4⁺ T cells are central to immunopathology. Initially, we assessed IL-7RA (CD127) expression on naive CD4⁺ T cells by transferring Thy1.1 CD62L^{hi} PLP-specific TCR transgenic CD4⁺ T cells into Thy1.2 recipients at day 10 post-EAE induction. We observed a significant increase in IL-7RA expression up to 48 hrs post-transfer, which was followed by progressive decline to background levels on day 6 (FIG. 18), suggesting that similar to the in vitro experiments the early stages of CD4⁺ T cell activation in vivo are also IL-7-dependent. In identical transfer studies but using CFSE-loaded allelically differing naive CD4⁺ T cells, we then examined whether brief administration of the anti-IL-7Rα antibody (2 injections/3 days) starting at the time of cell transfer would preferentially induce apoptosis of TCR-engaged autoreactive CD4⁺ T cells versus the polyclonal non-autoreactive host cells. In this case, there was a >90% reduction in the number of undivided (CFSE^{hi}) transgenic CD4⁺ T cells recovered from anti-IL-7Rα antibody-treated mice compared to controls (FIG. 16A), and most of the remaining CFSE^{hi} transgenic cells were preapoptotic (FIG. 16B). Parallel control transfer experiments of CFSE-labeled transgenic CD4⁺ T cells into non-PLP immunized SJL hosts showed no reduction of these cells following anti-IL-7Rα antibody treatment (FIG. 16C). Since IL-7RA expression was reacquired by CD4⁺ T cells several days after in vitro activation (FIG. 19), we examined whether autoreactive effector CD4⁺ T cells are also IL-7-dependent. Here, we transferred naive PLP-specific CD4⁺ TCR transgenic T cells (Thy1.1) into recipients (Thy1.2) simultaneous with EAE induction (day 0). After allowing for autoreactive T cell expansion and early disease manifestations (day 10; EAE severity score ≧1), we administered anti-IL-7Rα or control antibodies (3 injections/5 days). There was a significant reduction of CNS-infiltrating transgenic CD4⁺ T cells in the anti-IL-7Rα antibody-treated mice, and fewer of these cells expressed IL-2, IL-17, IFN-γ, and TNF-a (Table 1). In addition, IL-7Rα blocked mice showed a >65% reduction in the number of PLP-specific T cells in spleens (34±2×10⁴ vs. 11±1×10⁴; p<0.01) (FIG. 16D), with the majority of these cells undergoing apoptosis (75% Annexin V⁺ Thy1.1 versus 19% Thy1.2). These results reinforce the conclusion that recently activated naive and effector autoreactive CD4⁺ T cells are critically dependent on IL-7 during early and later stages of expansion and preferentially undergo apoptosis if deprived of this essential signal. They also indicate that elimination of PLP-specific recently activated CD4⁺ T cells in anti-IL-7Rα antibody-treated mice is not mediated by indirect effects such as opsonization, phagocytosis, ADCC, or complement-mediated lysis.

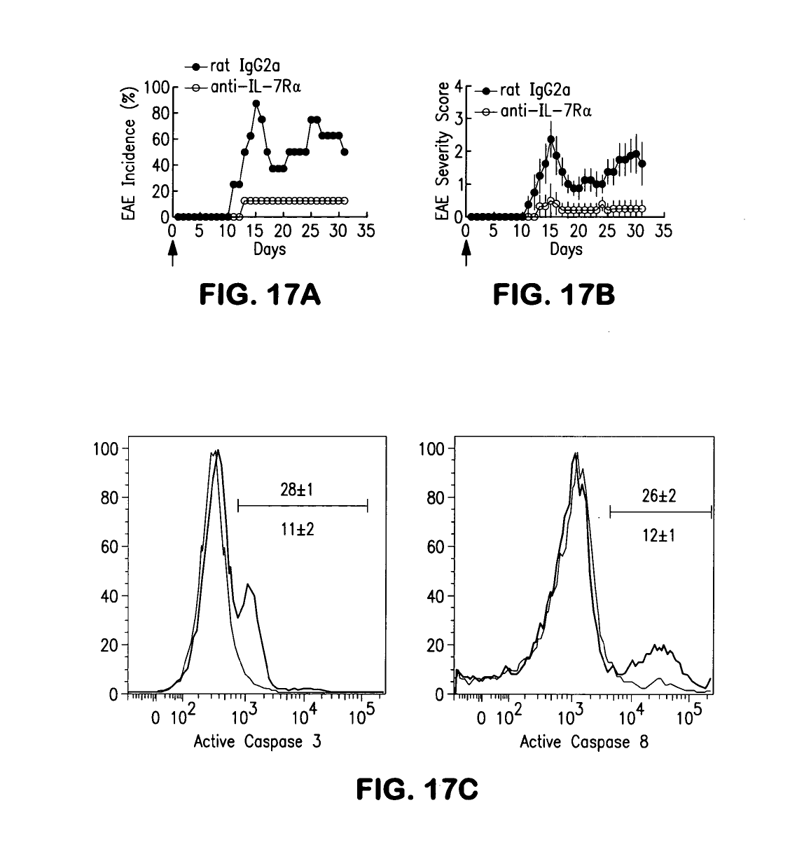

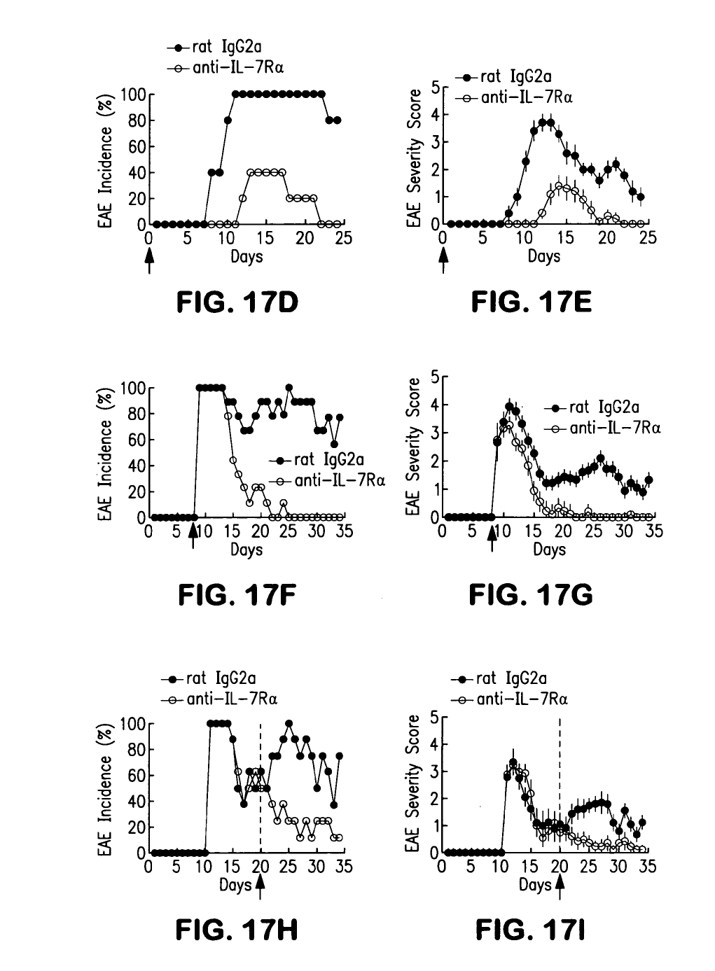

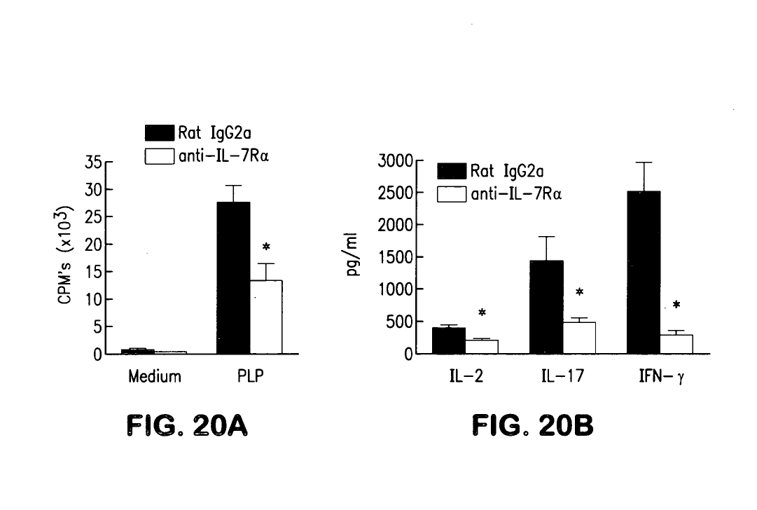

With these results, we proceeded to examine whether IL-7Rα blockade would be effective in reducing long-term manifestations of EAE, including relapses which occur in MS. We assessed the effectiveness of IL-7Rα blockade in the more clinically relevant relapsing/remitting model of EAE (R-EAE) in which both T_{H}1 and T_{H}17 CD4⁺ T cells have been implicated. We initially administered anti-IL-7Rα or control antibody (200 μg every 3 days i.p) simultaneously with immunization of SJL mice with PLP₁₃₉₋₁₅₁-peptide in adjuvant, and observed delayed onset as well as decreased incidence and severity (>5-fold) of disease in the anti-IL-7Rα-antibody-treated group compared to controls (FIGS. 4A and 4B). After weeks of antibody treatment, there was also a striking decrease in CNS infiltrating mononuclear cells (CD4⁺ and CD8⁺ T cells >20-fold, B cells >100-fold, monocytes >4-fold, and DCs >40-fold; Table 2), a higher percentage of apoptotic CD4⁺ T cells in the periphery (Annexin V⁺ 32±3% vs. 12±4% and activation of caspases 3 and 8 (FIG. 17C)), decreased in vitro PLP reactivity, and reduced IL-2, IFN-γ, and IL-17 production (FIG. 20). Consistent with the known IL-7 requirement for T cell development and survival, this treatment also resulted in significant reductions of thymic T cell subsets and peripheral naïve (CD44^{lo-int}CD62L^{hi}) CD4⁺ and CD8⁺ T cells (Table 2), whereas regulatory T cells (CD4⁺CD25⁺FoxP3⁺) with low IL-7RA expression, were unaffected (not shown). Blockade of IL-7RA was also effective in reducing EAE incidence and severity (>4-fold) when polarized T_{H}1-type PLP-specific CD4⁺ T cells were transferred into naive SJL recipients (FIGS. 17D and 17E), indicating that conversion to T_{H}17 cells, as proposed by others, is not required for this efficacious effect. More importantly, we found that IL-7RA blockade was effective when applied after the first signs of disease (day 8) (FIGS. 4F-4G) or at the early stages of relapse (day 20) (FIGS. 4H and 4I) with pathologic manifestations rapidly resolving without further recurrence. The overall results suggest that IL-7RA blockade can be effective in MS when applied at both early and late stages, particularly as a short-term treatment to avert relapses.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

TABLE 1
PLP-specific TCR transgenic CD4⁺ T cells producing
proinflammatory cytokines are reduced in the CNS
following anti-IL-7Rα antibody therapy in R-EAE.

| | Total | | | | |
|---|---|---|---|---|---|
| | Mononuclear | CD4⁺ (×10⁴) | | | |
| | Cells (×10⁶) | IL-2⁺ | IL-17⁺ | IFN-γ⁺ | TNF-α⁺ |
| Rat IgG2a | 1.3 ± 0.2 | 5 ± 1 | 4 ± 1 | 6 ± 1 | 4 ± 1 |
| anti- | 0.6 ± 0.1 | 1.1 ± 0.3 | 1.3 ± 0.3 | 1.6 ± 0.2 | 1.7 ± 0.3 |
| IL-7Rα | | | | | |
| p-value | 0.01 | 0.004 | 0.02 | 0.02 | 0.02 |

Recipient Thy1.2 SJL mice (6-8 mice/group) were challenged with PLP₁₃₉₋₁₅₁ in CFA on day 0. PTX was given on days 0 and 2. Naive PLP-specific T cells (3.5 × 10⁶ cells/mouse) from PLP₁₃₁₋₁₅₁ Thy1.1 TCR transgenic mice were adoptively transferred on day 0. Ten days later anti-IL-7Rα antibody was administered as described in Methods and mice sacrificed 5 days later. PLP transgenic T cells were isolated from the CNS, stimulated with PLP₁₃₁₋₁₅₉ (10 μg/ml) peptide plus IL-2 (5 ng/ml) for 5 hrs in the presence of monensin, surface stained, fixed, permeabilized, and intracellularly stained for the indicated cytokines. Results are total numbers ± SEM. P ≦ 0.05 is considered significant.

TABLE 2
Enumeration of immune cell numbers in CNS, thymus,
and spleen in anti-IL-7Rα-treated R-EAE mice.

| CNS (×10⁴) | CD45⁺ | CD4⁺ | CD8⁺ | B220⁺ | CD11b⁺ | CD11c⁺ |
|---|---|---|---|---|---|---|
| Rat IgG2a | 24.9 ± 5.8 | 6.8 ± 1.8 | 1.3 ± 4.2 | 3.6 ± 1.5 | 8.7 ± 2.9 | 4.5 ± 1.0 |
| anti-IL-7Rα | 3.6 ± 1.2 | 0.34 ± 0.2 | 0.6 ± 0.4 | 0.4 ± 0.2 | 1.9 ± 3.2 | 0.1 ± 0.05 |
| p-value | 0.01 | 0.01 | 0.01 | 0.03 | 0.03 | 0.002 |

| Thymus (×10⁶) | DN | DP | CD4⁺CD8⁻SP | CD4⁻CD8⁺ SP |
|---|---|---|---|---|
| Rat IgG2a | 4.2 ± 1.5 | 6.6 ± 1.2 | 12.5 ± 3.9 | 5.1 ± 1.53 |
| anti-IL-7Rα | 0.5 ± 0.25 | 1 ± 0.34 | 0.9 ± 0.25 | 0.5 ± 0.15 |
| p-value | 0.04 | 0.04 | 0.01 | 0.01 |

| Spleen (×10⁶) | Total CD4 | CD4⁺CD62L^{hi}CD44^{lo} | Total CD8 |
|---|---|---|---|
| Rat IgG2a | 49.8 ± 7.9 | 39.2 ± 4.6 | 24.9 ± 3.7 |
| anti-IL-7Rα | 26.2 ± 5.1 | 19.7 ± 2.2 | 11.0 ± 1.9 |
| p-value | 0.03 | 0.01 | 0.01 |

Female SJL mice (8-10/group) were immunized s.c. with PLP₁₃₉₋₁₅₁ in CFA on day 0 and PTX given on days 0 and 2. Anti-IL-7Rα or rat IgG2a isotype control antibody injections (both given i.p. at 200 μg/injection three times per week) began on day 0. Mice were sacrificed following EAE onset and indicated tissues examined for immune cell numbers and subtypes. Representative results are from one experiment of 3 independent experiments and are given as total numbers ± SEM. P ≦ 0.05 is considered significant.

1. A method of treating or alleviating the symptoms of a disorder associated with a hyperactive immune system in a subject, comprising administering to the subject a therapeutically effective amount of a compound that inhibits or suppresses IL-7 signaling or IL-15 signaling, thereby treating or alleviating the symptoms of a disorder associated with a hyperactive immune system in the subject.

2. The method of claim 1, wherein the compound inhibits or suppresses IL-7 signaling.

3. The method of claim 1, wherein the compound down-regulates expression or cellular level of IL-7.

4. The method of claim 3, wherein the compound is an inhibitory polynucleotide.

5. The method of claim 1, wherein the compound blocks signaling activities of IL-7 receptor.

6. The method of claim 5, wherein the compound is an antagonist antibody specific for IL-7Rα.

7. The method of claim 1, wherein the disorder associated with hyperactive immune response is an autoimmune disease.

8. The method of claim 7, wherein the autoimmune disease is lupus or multiple sclerosis.

9. A method of inhibiting activation of CD4⁺ T cells in a subject, comprising administering to the subject a therapeutically effective amount of a compound that inhibits or suppresses IL-7 signaling, thereby inhibiting activation of CD4⁺ T cells in the subject.

10. The method of claim 9, wherein the compound down-regulates expression or cellular level of IL-7.

11. The method of claim 10, wherein the compound is an inhibitory polynucleotide.

12. The method of claim 9, wherein the compound blocks signaling activities of IL-7 receptor.

13. The method of claim 12, wherein the compound is an antagonist antibody specific for IL-7Rα.

14. The method of claim 9, wherein the subject suffers from an inflammatory disorder.

15. The method of claim 14, wherein the inflammatory disorder is multiple sclerosis.

16. The method of claim 15, wherein the treatment is to prevent relapse of multiple sclerosis in the subject.

17. A method of inhibiting proliferation of an overactivated T cell in a subject, comprising administering to the subject a therapeutically effective amount of a compound that inhibits or suppresses IL-7 signaling or IL-15 signaling, thereby inhibiting proliferation the overactivated T cell in the subject.

18. The method of claim 17, wherein the compound inhibits or suppresses IL-7 signaling.

19. The method of claim 18, wherein the compound down-regulates expression or cellular level of IL-7.

20. The method of claim 19, wherein the compound is an inhibitory polynucleotide.

21. The method of claim 19, wherein the compound is a siRNA specific for IL-7.

22. The method of claim 18, wherein the compound blocks signaling activities of IL-7 receptor.

23. The method of claim 22, wherein the compound is an antagonist antibody specific for IL-7Rα.

24. The method of claim 17, wherein the subject suffers from an autoimmune disease.

25. The method of claim 24, wherein the autoimmune disease is lupus or multiple sclerosis.

26. The method of claim 17, wherein the overactivated T cell is CD4⁻CD8⁻ T cell.
